(19) United States
(12) Patent Application Publication
(10) Pub. No.: US 2023/0039279 A1
(43) Pub. Date: Feb. 9, 2023

(54) INFLAMMATION-RESPONSIVE ANTI-INFLAMMATORY HYDROGELS

(71) Applicant: Nanyang Technological University, Singapore (SG)

(72) Inventors: Thuy Tram DANG, Singapore (SG); Tri Dang NGUYEN, Singapore (SG); Hsin-Yueh NG, Singapore (SG)

(21) Appl. No.: 17/782,908

(22) PCT Filed: Dec. 7, 2020
(86) PCT No.: PCT/SG2020/050723

§ 371 Date: Jun. 6, 2022

(30) Foreign Application Priority Data
Dec. 6, 2019 (SG) 10201911767R

(51) Int. Cl. A61K 47/65; A61K 9/50; A61K 9/06; A61K 47/60; A61K 9/70

(52) U.S. Cl. CPC A61K 47/65; A61K 9/50; A61K 9/06; A61K 47/60; A61K 9/7023

(57) ABSTRACT

The present invention relates generally to the field of protease-responsive drug delivery hydrogels, uses thereof, and related methods of their production. More particularly, the invention relates to hydrogels which release anti-inflammatory agents upon reaction with inflammation-related proteases.

### CROSS-REFERENCE TO RELATED APPLICATIONS

The present application is a filing under 35 U.S.C. 371 as the National Stage of International Application No. PCT/SG2020/050723, filed Dec. 7, 2020, entitled “INFLAMMATION-RESPONSIVE ANTI-INFLAMMATORY HYDROGELS,” which claims priority to Singapore Application No. SG 10201911767R filed with the Intellectual Property Office of Singapore on Dec. 6, 2019, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4373-17500_SP101987USZBD_Sequence_Listing; created on Jun. 3, 2022; and having a files size of 44 KB.

The information in the Sequence Listing is incorporated herein in its entirety for all purposes.

### FIELD OF THE INVENTION

The present invention relates generally to the field of protease-responsive drug delivery hydrogels, use thereof, and related methods of their production. More particularly, the invention relates to hydrogels which release anti-inflammatory agents upon reaction with inflammation-related proteases.

### BACKGROUND OF THE INVENTION

Inflammation is a sequence of biological reactions mounted by the host immune system to remove harmful stimuli and restore a damaged tissue to its pre-injury condition [Serhan, C. N. et al. Fundamentals of Inflammation, Cambridge University Press, Cambridge, (2010)]. An acute inflammatory response is essential to eliminate harmful stimuli and restore cellular homeostasis after tissue injury [Serhan, C. N. et al. Fundamentals of Inflammation, Cambridge University Press, Cambridge, (2010)]. In contrast, undesirable persistence of leukocyte activity results in excessive inflammation associated with chronic tissue damage [Serhan, C. N. et al. Fundamentals of Inflammation, Cambridge University Press, Cambridge, (2010)]. This chronic condition is often encountered in many pathological conditions such as rheumatoid arthritis, chronic diabetic ulcers, inflammatory bowel diseases (IBDs) and chronic obstructive pulmonary diseases [Serhan, C. N. et al. Fundamentals of Inflammation, Cambridge University Press, Cambridge, (2010)].

Systemic administration of anti-inflammatory therapeutics is a clinically accepted treatment paradigm to mitigate excessive inflammation in chronic diseases. Small molecule drugs such as nonsteroidal anti-inflammatory drugs (NSAIDs) and steroidal immuno-suppressants are empirically prescribed to patients with rheumatoid arthritis and IBDs based on their clinical symptoms. However, systemic administration of these drugs is also implicated in the occurrence of well-known side effects, which are associated with excessive dosage resulting from a lack of controlled drug release. For instance, systematically administered NSAIDs raise the risk of myocardial infarction, cerebrovascular accidents, and gastric ulceration. In addition, corticosteroids result in severe drug-induced complications such as osteonecrosis, glaucoma, and opportunistic infection when prescribed over an extended duration.

To improve spatiotemporal control of drug release kinetics and minimize systemic toxicity, several drug delivery platforms have been designed for administration of anti-inflammatory therapeutics [Hämäläinen, M. et al. Basic & Clinical Pharmacology & Toxicology 112(5) 296-301 (2013)]. For example, encapsulating glucocorticoids in vesicular systems extended drug half-life and achieved a slower release of therapeutic drugs [Maestrelli, F. et al. Journal of Drug Delivery Science and Technology 32 192-205 (2016)]. Alternatively, covalent conjugation of a small molecule NSAID to nanoscale polymeric films has been reported as an alternative approach to substantially increase therapeutic payload and achieve gradual long-term release by hydrolysis of the drug-polymer ester linkage [Hsu, B. B. Proceedings of the National Academy of Sciences 111(33) 12175 (2014)]. However, these systems do not take into account the specific pathological conditions of the diseased tissues whose inflammatory characteristics necessitate the administration of anti-inflammatory therapeutics. Therefore, their drug release profiles do not match the biological requirements as the release is primarily dictated by the physiochemical characteristics of the delivery platforms such as polymer composition and drug loading capacity.

The inflammatory characteristics of the biological microenvironment at diseased tissues can be leveraged to design smart drug delivery systems which can be triggered by immunological cues. Particularly, published studies have established the upregulation of proteases, especially serine proteases and matrix metalloproteases (MMPs), in chronic inflammation, suggesting their potential as biochemical cues for therapeutic administration to modulate the inflammation cascade [Pham, C. T. N. The International Journal of Biochemistry & Cell Biology 40(6) 1317-1333 (2008)]. Compared to other stimuli such as pH, temperature, or redox, proteases still stand as the more specific biological cue as compared to the other stimuli, mainly due to the close relationship between dysregulation of proteases with a pathological condition. Moreover, other stimuli can be affected due to environmental conditions. For instance, body temperature can spike due to the humid weather condition and not due to the disease state. Despite proteases being a better biological cue, the potential of proteases as immunological cues for biologically-triggered drug delivery systems to modulate inflammation has largely remained unexplored. Recently, Joshi et al. leveraged the self-assembly of a small molecule amphiphile, triglycerol monostearate (TG-18), to physically entrap a corticosteroid in a hydrogel platform which could be triggered to release this drug upon exposure to increased arthritic flare activity [Joshi, N. et al. Nature Communications 9(1) 1275 (2018)]. Nonetheless, this reported drug-loaded hydrogel platform lacks a generalizable design framework, thus limiting the possibility of replacing its components to exploit alternative biological triggers. Specifically, drug release from this platform relies on the cleavage of ester bonds on the TG-18 backbone primarily by esterases, which are upregulated in inflammatory arthritis [Ravaud, P. et al. Rheumatology 41(7) 815-818 (2002)] but might not be a critical biological cue in other inflammatory diseases. Non-enzymatic hydrolysis of these ester bonds in the low pH environment associated with inflammatory conditions [Bellocq, A., et al. Journal of Biological Chemistry 273(9) 5086-5092 (1998); Riemann, A. et al. Molecular Basis of Disease 1862(1) 72-81 (2016)] might also result in undesirable non-specific drug release.

Therefore, a protease-triggered drug delivery platform that is (1) modular in design, (2) immuno-compatible and (3) versatile for both injectable and topical administration at room temperature still represents an unmet need to address limitations of the existing delivery systems. Firstly, physical entrapment of a drug in a particulate domain such as liposome or polymeric microparticles embedded in a protease-triggered delivery systems might be associated with a diffusion-driven basal drug release. This basal release might be desirable for management of chronic inflammatory conditions that requires a protease-triggered increased dosage when the condition is suddenly exacerbated due to infection onset or arthritic flares. However, this basal release is not always desirable in all inflammation-associated conditions, particularly in immuno-compromised patient or patients on immuno-suppressant drugs or for management of acute injury where some extent of inflammation is required for normal healing. An alternative design of protease-triggered delivery system which eliminates or minimizing this basal drug release is also desirable for conditions in which the drug-administered site undergoes a transition from physiological state requiring no drug to highly inflammatory pathological states such as sudden onset of bacterial infection on acute wounds or flare-up of seborrheic dermatitis.

Secondly, leveraging a single protease as a biochemical stimulus for triggering drug release in the management of inflammation-associated pathology can partially help to tailor the dosage to the inflammation condition of the diseases. However, multiple proteases might be upregulated in a pathological inflammatory condition. Therefore, utilizing a subset of proteases instead of a single protease can increase the specific association of protease activity with disease-specific condition to achieve drug release kinetics specifically tailored to the inflammation-associated disease of interest. Thus, there also remains a substantial need for the development of a drug delivery system whose drug release is triggered by two or more protease stimuli (or plural protease responsivity) to achieve enhanced specificity.

### SUMMARY OF THE INVENTION

The present invention provides an inflammation-responsive drug delivery platform comprising of (1) drug-loaded domains (either particles encapsulating anti-inflammatory drugs or conjugated anti-inflammatory drug) with a tailored basal drug release profile and/or (2) a proteases-cleavable hydrogel domain. This invention provides a drug delivery platform which can be customized to cope with an inflammatory disease by changing the configuration of its drug-loaded domain and/or adjusting the plural sensitivity of its protease-triggered domain to tailor its responsiveness and specificity to the disease of interest.

According to a first aspect of the invention, there is provided a drug-loaded protease-responsive hydrogel comprising;

a) a drug encapsulated in particles;
b) a polymer building block comprising of a multi-arm-polyethylene glycol (PEG) with functional moiety; and
c) a bis-functional protease-sensitive crosslinker comprising a protease-cleavable substrate flanked by two spacer sequences containing functional moieties;
wherein said polymer building block of b) forms a gel in the presence of the protease-cleavable crosslinker of c) to entrap the particles of a).

In some embodiments the drug-loaded protease-responsive hydrogel further comprises:

a) at least a second bis-functional protease-sensitive crosslinker which comprises a protease-cleavable substrate, sensitive to a different protease to that of said crosslinker of c), flanked by spacer sequences containing functional moieties; and/or

b) at least a further bis-functional protease-resistant crosslinker which comprises a protease-resistant substrate.

The particles may be comprised of any suitable material that can carry and release a drug (such as a small molecule, therapeutic peptide, protein, mRNA, or the like) and be entrapped by the gel formed by the polymer building block and crosslinker. For example, the particles may be silica, liposomes, siRNA complexes or polymeric material. Particles can be made using well-known prior art methods such as emulsion, electrospraying, electrostatic complexation, flow-focusing method, etc., [Abdelaziza, Hadeer M. et al., Journal of Controlled Release 269 374-392 (2018)].

In some embodiments the drug is encapsulated in particles comprising a polymeric material selected from the group comprising polycaprolactone, poly (methacrylic acids), polylactic acids, polyvinylpirrolidone, poly(lactic-co-glycolic acid) (PLGA) and gelatin. Preferably, the particles are microparticles and/or nanoparticles, preferably having a diameter in the range of about 10 nm to about 100 μm.

In some embodiments the polymer building block comprises a multi-arm-PEG-vinyl sulfone or multi-arm-PEG-maleimide or multi-arm-PEG-azide or multi-arm-PEG-alkyne. Advantageously, the sulfone moiety interacts with a cysteine moiety on an arm of the crosslinker.

The invention also embodies a drug-loaded protease-responsive hydrogel that does not require encapsulation of the drug in particles for containment until released by said protease.

According to a second aspect of the invention, there is provided a drug-loaded protease-responsive hydrogel comprising;

a) a drug covalently conjugated to a protease-cleavable peptide anchor having a functional moiety;
b) a polymer building block comprising a multi-arm-PEG polymer having at least one functional moiety; and
c) a bis-functional crosslinker comprising a peptide substrate flanked by spacer sequences containing functional moieties;

wherein the functional moiety of the peptide anchor covalently links the drug to an arm of the multi-arm PEG polymer, and wherein a functional moiety of said polymer building block covalently links to said moiety of said bis-functional crosslinker to form a gel.

The arrangement of peptide anchor and crosslinker provides flexibility and tuning of the release profile of the drug-loaded hydrogel, whereby the release of the drug may be sensitive to one or more different proteases.

Advantageously, the drug-conjugated domain minimizes basal release of the drug.

In some embodiments:

a) said crosslinker is not cleavable to a protease; or
b) said peptide anchor is cleavable to a protease and said crosslinker is cleavable to the same or different protease; and/or
c) said drug-loaded hydrogel comprises a plurality of crosslinkers, one or more of which are cleavable to different proteases.

Advantageously, a desired peptide anchor consists of a protease-cleavable spacer sequence containing a functional moiety, which comprises at least 4 amino acids.

Advantageously, a crosslinker consists of a protease-cleavable substrate sequence flanked by spacer sequences containing functional moieties, each of which comprises at least 4 amino acids.

The non-cleavable crosslinker is used to control the diffusion of the enzyme into the gel network and hence help to tune the release profile.

In some embodiments the drug may be a small molecule, siRNA, aptamer or therapeutic peptide or protein.

Advantageously, the combination of peptide sequences, which are the key component of the protease-triggered domain, provide a fast water-based gelation and better specifically-triggered release upon exposure to more than one disease-specific proteases.

In some embodiments the polymer building block comprises a multi-arm-PEG-vinyl Maleimide. The amount of drug loaded onto the protease-responsive hydrogel may be controlled by the amount or concentration of multi-arm-PEG polymer used.

In some embodiments the weight ratio of the drug-loaded protease-responsive hydrogel is from about 2 w/v % to about 12 w/v %, preferably from about 3 w/v % to about 10 w/v %. Preferably the hydrogel is a multi-arm-PEG-vinyl sulfone or a multi-arm-PEG-vinyl Maleimide or a multi-arm-PEG-alkyne or a multi-arm-PEG-azide.

It would be understood that the number of arms on the multi-arm-PEG polymer will have an effect on the amount of drug that can be conjugated and also on the degree of crosslinking and gel formation.

In some embodiments of the drug-loaded protease-responsive hydrogel of any aspect of the invention, the multi-arm PEG polymer has 3 to 8 arms.

In some embodiments of the drug-loaded protease-responsive hydrogel of any aspect of the invention, the drug is anti-inflammatory.

In some embodiments of the drug-loaded protease-responsive hydrogel of any aspect of the invention, the protease is upregulated during inflammation and is selected from the group comprising matrix metalloproteinases and serine proteases.

In some embodiments of the drug-loaded protease-responsive hydrogel of any aspect of the invention, the drug is a steroidal anti-inflammatory drug or a non-steroidal anti-inflammatory drug (NSAID), or derivatives thereof. The drug may be a steroidal anti-inflammatory drug such as Dexamethasone, Fludrocortisone, Methylprednisolone, Prednisolone, Prednisone or Hydrocortisone, or derivatives thereof. Glucocorticoids can be oxidized to add a carboxylic functional group which allows these drugs to be conjugated to the peptide anchors of the invention. Preferably the drug is a NSAID, such as Ibuprofen, Ketoprofen, Diclorofenac, Sunlindac, Piroxicam, or Celecoxib, or derivatives thereof.

In some embodiments of the drug-loaded protease-responsive hydrogel of any aspect of the invention, the said flanking spacer sequences comprise at least one Cysteine and/or Lysine residue and/or azide- or alkyne-containing unnatural amino acid which are required to react with the functional moiety of the multi-arm PEGs to induce gelation. The spacer may have 1-6 amino acids. The remaining residues can be any of the amino acids, preferably amino acid with charged side groups. Specifically, positive charges amino acids (e.g., arginine, R) close to thiol moieties of cysteines increase the crosslinking rate while negative charges (e.g., aspartic acid, D) decelerated this reaction. The spacer may have 1-6 amino acids, In some embodiments, the flanking spacer sequence (“SPACER”) may be of the formula GX₁X₂X₃, (SEQ ID NO: 33) wherein each of X₁, X₂ and X₃ is independently Glycine, Cysteine, Aspartic acid, or Arginine and/or the reverse sequence thereof. In some embodiments, the said flanking spacer sequences are selected from the group comprising GRCR (SEQ ID NO; 1), GCRG (SEQ ID NO: 2), GRCD (SEQ ID NO: 3), GCDR (SEQ ID NO: 4), GCDG (SEQ ID NO: 5), GDCD (SEQ ID NO: 6), GCDD (SEQ ID NO: 7), GCRD (SEQ ID NO: 8) and GCRR (SEQ ID NO: 9).

When first and second spacers are used, one at each end of a peptide substrate, the second spacer sequence may be the reverse of the first spacer sequence and may be of the formula X₃ X₂ X₁ G (SEQ ID NO: 34). This reversed spacer sequence may be referred to as a “RECAPS” and, for example, be the reverse sequence of a spacer selected from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, ID NO: 8 and SEQ ID NO: 9.

In some embodiments of the drug-loaded protease-responsive hydrogel of any aspect of the invention the protease-cleavable substrate is sensitive to a protease selected from the group comprising matrix metalloproteinases, such as metalloproteinase-9 (MMP-9), MMP-2, MMP-7, MMP-12 etc., cathepsins, such as Cathepsin K, Cathepsin B, Cathepsin S, etc., human neutrophil elastase (HNE), caspases and urokinases.

In some embodiments, the protease-cleavable substrate is selected from the group comprising MMP-9 substrates comprising the amino acid sequence set forth in KGPRSLSGK (SEQ ID NO: 30), GPRSLSG (SEQ ID NO: 10), LGRMGLPGK (SEQ ID NO: 11), AVRWLLTA (SEQ ID NO: 12) or GPQGIWGQ (SEQ ID NO: 13); HNE substrates comprising APEEIMDRQ (SEQ ID NO: 14) or PMAVVQSVP (SEQ ID NO: 15); Cathepsin B substrates comprising GRRGLG (SEQ ID NO: 16) or DGFLGDD (SEQ ID NO: 17) or a combination thereof.

According to a third aspect of the invention, there is provided a composition comprising the drug-loaded protease-responsive hydrogel of any aspect of the invention formulated for injection or topical administration.

The drug-loaded inflammation-responsive hydrogel can be incorporated onto a polymeric dressing to form a composite dressing for wound management.

According to a fourth aspect of the invention, there is provided a dressing comprising the drug-loaded protease-responsive hydrogel of any aspect of the invention.

According to a fifth aspect of the invention, there is provided of the drug-loaded protease-responsive hydrogel of any aspect of the invention or a composition of the invention as an injectable or topical dressing for treating a subject in need thereof.

According to a sixth aspect of the invention, there is provided a method of treatment comprising administering to a subject in need of such treatment an efficacious amount of the drug-loaded protease-responsive hydrogel of any aspect of the invention or a composition of the invention. In some embodiments the administration is by injection or topical application to the subject. In some embodiments, the treatment is for inflammation-associated diseases such as chronic wounds, inflammatory bowel diseases, arthritis and potentially infection-related conditions for which inflammation management is desirable.

According to a seventh aspect of the invention, there is provided a kit comprising:

a) a drug encapsulated in particles;

b) a polymer building block comprising of a multi-arm-polyethylene glycol (PEG) with functional moiety; and

c) a bis-functional protease-sensitive crosslinker comprising a protease-cleavable substrate flanked by two spacer sequences containing functional moieties, wherein a)-c) are as defined in any one of the previous aspects; or

a) a drug covalently conjugated to a protease-cleavable peptide anchor having a functional moiety;

b) a polymer building block comprising a multi-arm-PEG polymer having at least one functional moiety; and

c) a bis-functional crosslinker comprising a peptide substrate flanked by spacer sequences containing functional moieties, wherein a)-c) are as defined in any one of the previous aspects.

In some embodiments, the kit comprises the drug-loaded protease-responsive hydrogel of any aspect of the invention or a composition of any aspect of the invention.

According to an eighth aspect of the invention, there is provided a method of manufacturing a drug-loaded protease-responsive hydrogel comprising the steps:

a) mixing a polymer building block comprising of a multi-arm-polyethylene glycol (PEG) with functional moiety with drug-loaded particles;
b) mixing a bis-functional protease-sensitive crosslinker, comprising a protease-cleavable substrate flanked by spacer sequences containing functional moieties, with drug-loaded polymeric particles;
c) mixing together the mixtures of a) and b);
wherein said polymer building block of a) forms a gel in the presence of the protease-cleavable crosslinker of b) to entrap the drug-loaded particles.

The particles may be comprised of any suitable material that can carry and release a drug (such as a small molecule, therapeutic peptide, protein, mRNA, or the like) and be entrapped by the gel formed by the polymer building block and crosslinker. For example, the particles may be silica, liposomes, siRNA complexes or polymeric material. Particles can be made using well-known prior art methods such as emulsion, electrospraying, electrostatic complexation, flow-focusing method, etc., [Abdelaziza, Hadeer M. et al., Journal of Controlled Release 269 374-392 (2018)].

In some embodiments the drug is encapsulated in particles comprising a polymeric material selected from the group comprising polycaprolactone, poly (methacrylic acids), polylactic acids, polyvinylpirrolidone, poly(lactic-co-glycolic acid) (PLGA) and gelatin. Preferably, the particles are microparticles and/or nanoparticles, preferably having a diameter in the range of about 10 nm to about 100 μm.

According to a ninth aspect of the invention, there is provided a method of manufacturing a drug-loaded protease-responsive hydrogel comprising the steps:

a) mixing a drug which is covalently conjugated to a peptide anchor having a functional moiety with a polymer building block comprising a multi-arm-PEG polymer having at least one functional moiety, wherein the respective functional moieties of the peptide anchor and multi-arm-PEG polymer covalently bond to conjugate the drug to an arm of the multi-arm PEG polymer;
b) mixing the drug-polymer conjugate of a) with a bis-functional crosslinker comprising a peptide substrate flanked by spacer sequences containing functional moieties;
wherein a functional moiety of said polymer building block covalently links to said moiety of said bis-functional crosslinker to form a gel.

In some embodiments of the ninth aspect:

a) said peptide anchor is cleavable to a protease and said crosslinker is not cleavable to a protease; or
b) said peptide anchor is cleavable to a protease and said crosslinker is cleavable to the same or different protease; and/or
c) said drug-loaded hydrogel comprises a plurality of crosslinkers, one or more of which are cleavable to different proteases.

In some embodiments, the drug, the particles, the crosslinker, the cleavable anchor and/or the polymer building block are as defined in any aspect of the invention.

According to a tenth aspect of the invention, there is provided a method of manufacturing a composite dressing comprising a drug-loaded protease-responsive hydrogel of any aspect of the invention, comprising the steps;

a) preparing a mixture of a drug-encapsulated in particles and a bis-functional protease-sensitive crosslinker comprising a protease-cleavable substrate flanked by two spacer sequences containing functional moieties;
b) preparing a mixture of a drug-encapsulated in particles and a polymer building block comprising of a multi-arm-polyethylene glycol (PEG) with functional moiety;
c) mixing a) and b) together, depositing the mixture onto a dressing and allowing gelation.

In some embodiments, the dressing is an alginate wound dressing.

In some embodiments, the method further comprises step d), wherein the composite dressing is flash-frozen in liquid nitrogen and lyophilized to dryness.

In some embodiments of the method of manufacturing, the drug is a NSAID; the particle comprises poly(lactic-co-glycolic acid) (PLGA); the crosslinker and/or anchor are cleavable to a protease selected from the group comprising matrix metalloproteinases and serine proteases or combinations thereof; and the polymer building block comprises a 4- or 8-arm-PEG-vinyl sulfone or a 4- or 8-arm-PEG-vinyl Maleimide or a 4 or 8-arm-PEG-azide or a 4 or 8-arm-PEG-alkyne.

Advantageously, the generalizable design framework enables the changes in choices and loading capacity of drugs while maintaining its structural and functional integrity.

Advantageously, immuno-compatible materials are utilized in the design of this delivery platform to potentially minimize adverse host response upon its administration in vivo.

Advantageously, this platform is versatile for both injectable and topical administration at room temperature.

### BRIEF DESCRIPTION OF THE FIGURES

FIG. 1 shows the formation of the modular particulate-based hydrogel GEL-iP and triggered release of drug-loaded particles in response to singular protease activity.

FIG. 2 shows an example of a hybrid particulate-based hydrogel illustrating successful gelation and MMP-9 triggered release of ibuprofen-loaded particles (ibu-PLGA particles). Addition of bis-cysteine peptide as a peptide crosslinker induced the gelation (vial A1). In the absence of this crosslinker, the gelation did not occur (vial A2). Gel dissolution due to MMP-9 activity (vial B1) caused the release of drug-loaded particles into the surrounding medium as observed in its optical microscope image (C1). Complete digestion of 200 μL of hybrid hydrogel was achieved after 5 days. Without MMP-9 activity, the gel remained intact (vial B2) and the drug-loaded particles were not observed in the surrounding medium (C2). (Scale bar: 50 μm).

FIG. 3 shows MMP-9 triggered the in vitro release of ibuprofen from the hybrid hydrogel GEL-iP. Exposing GEL-iP to MMP-9 ( [symbol] ) significantly boosted the cumulative drug release compared to the control ( [symbol] ). Addition of an MMP-9 inhibitor suppressed the release of ibuprofen ( [symbol] ). Slower release kinetics were observed from the non-cleavable hybrid hydrogel (scrGEL-iP, [symbol] ). Error bars represent s.e.m of n=4 replicates.

FIG. 4A-B shows the effect of MMP-9-triggered drug release from the hybrid hydrogel GEL-iP on macrophage proliferation. A) Schematic of releasate generation, collection, and treatment of seeded cells. B) Relative metabolic activity of macrophages after 72 hours of exposure to releasates generated from culture media only, freely dissolved drug, ibuprofen-free hybrid hydrogel GEL-P, GEL-iP, or scrGEL-iP in the presence or absence of MMP-9 and its inhibitor. Error bars represent s.e.m of n=4 replicates. p-values were determined by one-way ANOVA with Fisher LSD post-hoc analysis. (***), (****), and (ns) denote p<0.001, p<0.0001, and p≥0.05 (not significant) respectively. Ibu: ibuprofen; Blank PLGA particles: ibuprofen-free PLGA particles; ibu-PLGA particles: ibuprofen-loaded PLGA particles; GEL-P: PEG hydrogel crosslinked by cleavable peptide (1) (FIG. 14; GCRR-KGPRSLSGK-RRCG; SEQ ID NO: 18) and embedded with blank PLGA particles; GEL-iP: PEG hydrogel crosslinked by cleavable peptide (1) (FIG. 14; GCRR-KGPRSLSGK-RRCG; SEQ ID NO: 18) and embedded with ibu-PLGA particles; scrGEL-iP: PEG hydrogel crosslinked by scrambled peptide (GCRR-KSSRGGPLK-RRCG; SEQ ID NO: 29) and embedded with ibu-PLGA particles.

FIG. 5A-C shows in vivo evaluation of reactive oxygen species (ROS) activity induced by the hybrid hydrogel GEL-iP and its constituent materials in immuno-competent SKH-1E mice. A) Experimental design illustrating subcutaneous injections of 6 material formulations on the dorsal side of mice and subsequent quantification of ROS activity via bioluminescent imaging. B) Bioluminescent image of a representative mouse on day 3. C) Quantification of ROS activity indicating its reduction to background level after 5 days. Error bars represent s.e.m of n=6 injections. p-values were determined by one-way ANOVA with Fisher LSD post-hoc analysis. (*) and (ns) denote p<0.05 and p≥0.05 (not significant) respectively. Alginate gel: alginate hydrogel crosslinked by calcium chloride; PEG gel: PEG hydrogel crosslinked by cleavable peptide; GEL-P: PEG hydrogel crosslinked by cleavable peptide and embedded with PLGA particles; GEL-iP: PEG hydrogel crosslinked by cleavable peptide and embedded with ibu-PLGA particles.

FIG. 6A-C shows that dual proteases triggered the release of PLGA particles from the plural protease-cleavable hydrogel. A) Schematic of the mechanism of dual protease-responsive combinational hydrogel system. B) Photographs show the cleavability of H2-M2 combinational hydrogels over 24 hours (n=3). (i) Control, (ii) With HNE protease; (iii) With MMP-9 protease; and (iv) With both HNE and MMP-9 proteases. C) Quantification of the average number of PLGA particles released from combinational hydrogels in the presence of zero, single or dual proteases over 24 hours (n=3). Error bars represent s.e.m of n=3 replicates. p-values were determined by one-way ANOVA with Tukey post-hoc analysis. (***) and (ns) denote p≤0.001 and p≥0.05 (not significant) respectively.

FIG. 7A-C shows fabrication and protease-triggered in vitro release of ibuprofen from the composite dressing incorporating GEL-iP. A) Schematic of fabrication of the composite dressing from GEL-iP and Kaltostat® dressing. B) Schematic of MMP-9-triggered ibuprofen release from the composite dressing. C) Quantification of ibuprofen released from the composite dressing in response to MMP-9. Error bars represent s.e.m of n=4 replicates. p-value was determined by Student's t-test with Welch's correction. (**) denotes p<0.01.

FIG. 8A-C shows the design of an ibuprofen-conjugated MMP-9-cleavable hydrogel. A) Schematic of the conjugation and chemical structure of ibu-peptide conjugation drawn by ChemDraw®. B) MS spectrum of ibu-GPQGIWGQ-DRCG (SEQ ID NO: 19). C) Schematic and a typical example of the gelation of ibuprofen-conjugated MMP-9-cleavable hydrogel.

FIG. 9A-B shows the cleavability of Ibuprofen-conjugated MMP-9-triggered PEG hydrogel. A) MMP-9-induced digestion of the hydrogel system. B) schematic and MS spectrum of liberated ibuprofen caused by MMP-9.

FIG. 10A-B shows responsive release to inflammatory protease stimulus. A) The cumulative liberation of ibuprofen (LIbu) increased corresponding to the increase in the MMP-9 concentrations. Absence of diffusion-driven basal release in the absence of MMP9 protease (bottom line). B) The specificity of GPRSLSGRRCG (SEQ ID NO: 20) sensitivity to MMP-9 compared to Cathepsin B and HNE. Error bars represent standard error of the means of n=4 replicates.

FIG. 11A-C shows tunable drug loading and release rate. The release rate could be tuned by changing (A) crosslinkers (i.e. using the same anchor H while changing the crosslinker from xH to xM and control scrambled xM (i.e xM(scr): GCRR-SSRGGPL-RRCG, SEQ ID NO: 39) or (B) anchors (i.e. using the same crosslinker xH while changing the anchor from H to M, and control scrambled M (M(scr): SSRGGPL-RRCG, SEQ ID NO: 40). C) The amount of loaded drug could be improved by changing the number of PEG arms or PEG wt %. The arrow indicates an MMP-9 spike mimicking a sudden flare. Error bars represent standard error of the means of n=4 replicates. Legend label follows the format of ibu-anchor crosslinker i.e ibu-H_xH represents ibuprofen-conjugated hydrogel with anchor H and crosslinker xH

FIG. 12A-B shows a mouse model with different inflammatory severities in subcutaneous space. A) a schematic diagram of timeline. A photograph B) and fluorescent image C) show a representative set of mice with 3 severity levels. D) shows the quantification of fluorescent signals indicating the upregulation of MMPs activity while E) shows quantification of MMP-9 secretion using ELISA.

FIG. 13A-C shows inflammation-triggered drug release in subcutaneous space of SKH1-E mice. A) Experimental design illustrating subcutaneous inflammation creation followed by subcutaneous injections of drug-conjugated hydrogels on the dorsal side of mice and subsequent retrieval of gel blobs 12 hours post hydrogel injection. B) Photograph of three representative mouse skin tissues with gel blobs corresponding to 3 severity levels on day 3. C) Evaluation of percentage of drug release. Error bars represent s.e.m of n=8 mice.

FIG. 14A-B shows qualitative screening of multiple peptide crosslinkers, each comprising of a substrate and two similar spacers in the form of SPACER-SUBSTRATE-SPACER listed in Table 3. A) Gelation was confirmed when the mixture of PEG-VS, peptide crosslinker and ibuprofen-loaded particles stopped flowing despite gravity to form a white hybrid hydrogel at the bottom of the vial. B) Relative gelation rate was evaluated by comparing the duration taken for the liquid mixture to stop flowing. Gelation within 5 minutes was considered fast while gelation requiring more than 30 minutes was considered slow. C) The cleavability of each hybrid hydrogel was inspected by observing the amount of drug-loaded particles released into the surrounding medium under an optical microscope after MMP-9 exposure. (YES) indicates a significantly higher number of particles released in the presence of MMP-9, confirming hydrogel cleavability while (NO) indicates otherwise. (n.e) denotes a condition that was not evaluated.

FIG. 15A-E shows scanning electron microscope images of ibuprofen-loaded PLGA particles of different diameters. Different homogenizing speeds resulted in particles with approximate average diameters of 46 μm (A), 14 μm (B), 11 μm (C), 6 μm (D), and 4 μm (E). (All scale bars represent 20 μm).

FIG. 16 shows cumulative drug release from ibuprofen-loaded PLGA particles with different sizes. Larger particle diameters resulted in slower drug release kinetics. All error bars represent s.e.m of n=4 replicates.

FIG. 17A-B shows in situ formation of PEG gel crosslinked by MMP-9 cleavable peptide (1) (FIG. 14; GCRR-KGPRSLSGK-RRCG; SEQ ID NO: 18). A) Image of a dorsal side of a mouse with 2 lumps resulting from the subcutaneous injection of a precursor solution of PEG gel. B) Photograph of the excised skin tissue with crosslinked PEG gels at two injection sites 15 minutes post-injection, confirming the in-situ formation of PEG gel in subcutaneous space.

FIG. 18A-B shows post-injection appearance of injected materials in a representative mouse. A) Image of a mouse right after the subcutaneous injection of 6 material formulations (Alginate gel, PEG gel, ibu-PLGA particles, PLGA particles, GEL-P, and GEL-iP). B) Image of the hypodermal side of the excised skin containing 6 material formulations 5 days post-injection. Disappearance of PEG gel indicated its in vivo degradability.

FIG. 19 shows proteolytical functionalities of dual-responsive ibuprofen-conjugated PEG hydrogel. Exposing the gel to MMP-9 and HNE significantly boosted the cumulative drug release compared to that resulted from exposing the gel to either MMP-9 or HNE. The slowest release kinetics was observed from the gel immersed in protease-free buffer. Error bars represent s.e.m of n=4 replicates.

Bibliographic references mentioned in the present specification are for convenience listed at the end of the examples. The whole content of such bibliographic references is herein incorporated by reference.

### DETAILED DESCRIPTION OF THE INVENTION

### Definitions

Certain terms employed in the specification, examples and appended claims are collected here for convenience.

As used in the specification and the appended claims, the singular forms “a,” “an” and “the” include plural referents unless the context clearly dictates otherwise.

As used herein, ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that each unit between two particular units are also disclosed. For example, if 3 and 10 are disclosed, then 4, 5, 6, 7, 8, and 9 are also disclosed.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

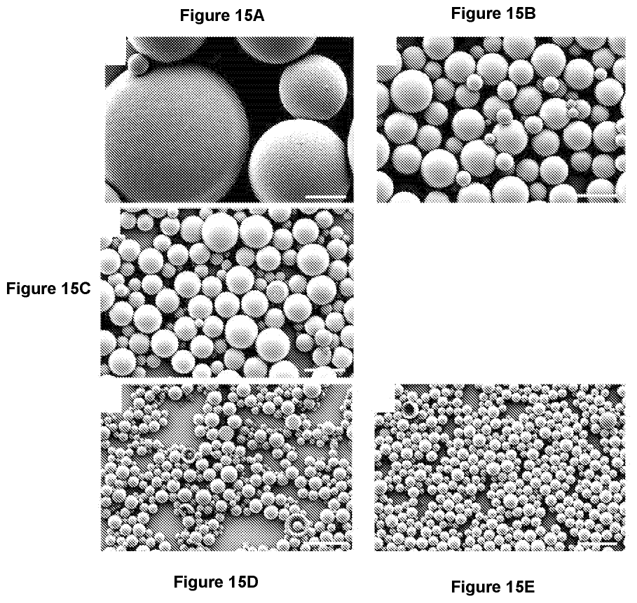

As used herein, the terms “polypeptide”, “peptide” or “protein” refer to one or more chains of amino acids, wherein each chain comprises amino acids covalently linked by peptide bonds, and wherein said polypeptide or peptide can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. A “polypeptide”, “peptide” or “protein” can comprise one (termed “a monomer”) or a plurality (termed “a multimer”) of amino acid chains. The term “particle” is used herein to broadly describe a material that encapsulates a drug and may be comprised of any suitable material that can carry and release the drug (such as a small molecule, therapeutic peptide, protein, mRNA, or the like) and be entrapped by the gel formed by the polymer building block and crosslinker. For example, the particles may be silica, liposomes, siRNA complexes or polymeric material. Particles can be made using well-known prior art methods such as emulsion, electrospraying, electrostatic complexation, flow-focusing method, etc., [Abdelaziza, Hadeer M. et al., Journal of Controlled Release 269 374-392 (2018)]. Polymeric particles are generally spheroidal in shape as shown in FIG. 15. Preferred particle sizes for use in the invention are microparticles and/or nanoparticles, having diameters in the nm and μm range. Preferably the particles have diameters in the range of 10 nm to 100 μm.

The term “polymer” or “biopolymer” is defined as a substance with repeated molecular units to become polymeric. The polymer may be a biocompatible polymer, selected from the group comprising polysaccharide (e.g. agarose, dextran), polyphosphazene, poly(acrylic acids), poly(methacrylic acids), copolymers of acrylic acid and methacrylic acid, poly(alkylene oxidase), poly(vinyl acetate), polyvinylpyrrolidone (PVP), their derivatives and copolymers and blends thereof. In respect of drug-encapsulated polymeric particles, the polymer may be, for example, selected from the group comprising polycaprolactone, poly (methacrylic acids), polylactic acids, polyvinylpirrolidone, poly(lactic-co-glycolic acid) (PLGA) and gelatin. The polymer may be a flexible polymer that is also mechanically and structurally stable and suitable for injection, transplantation or implantation (e.g. subcutaneous transplantation or implantation). The polymer may or may not be biodegradable. Polymer building blocks of the invention generally comprise a plurality of arms which have functional moieties that can interact with a functional moiety on a crosslinker to form a gel. Preferred multi-arm building blocks include multi-arm-PEG-vinyl sulfone, multi-arm-PEG-vinyl Maleimide, multi-arm-PEG-zide and multi-arm-PEG-alkyne, more particularly those with 4 or 8 arms.

The term “subject” is herein defined as vertebrate, particularly mammal, more particularly human. For purposes of research, the subject may particularly be at least one animal model, e.g., a mouse, rat and the like. In particular, for treatment or prophylaxis of a disease, such as an inflammatory disease, the subject may be a human.

The term ‘treatment’, as used in the context of the invention refers to prophylactic, ameliorating, therapeutic or curative treatment.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

While aspects of the present invention will be described in conjunction with the embodiments provided herein, it will be understood that they are not intended to limit the present invention to these embodiments. On the contrary, the present invention is intended to cover alternatives, modifications and equivalents to the embodiments described herein, which are included within the scope of the present invention as defined by the appended claims. Furthermore, in the following detailed description, specific details are set forth in order to provide a thorough understanding of the present invention. However, it will be recognized by an individual having ordinary skill in the art, i.e. a skilled person, that the present invention may be practiced without specific details, and/or with multiple details arising from combinations of aspects of particular embodiments. In a number of instances, known systems, methods, procedures, and components have not been described in detail so as to not unnecessarily obscure aspects of the embodiments of the present invention.

### EXAMPLES

A person skilled in the art will appreciate that the present invention may be practiced without undue experimentation according to the methods given herein. The methods, techniques and chemicals are as described in the references given or from protocols in standard biotechnology and molecular biology text books. Standard molecular biology techniques known in the art and not specifically described were generally followed as described in Sambrook and Russel, Molecular Cloning: A Laboratory Manual, Cold Springs Harbor Laboratory, New York (2001).

### Example 1: Materials and Methods for Fabrication of Protease-Responsive Particulate-Based Drug-Encapsulated Hybrid Hydrogels

### 1.1 Fabrication and Characterization of PLGA Particles

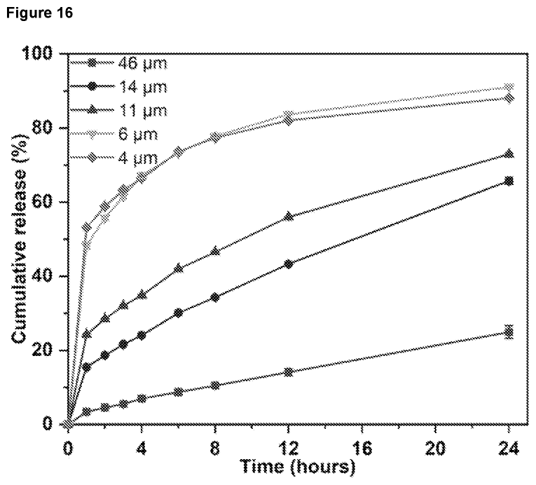

Particles with or without ibuprofen were fabricated via an oil-in-water emulsification method with poly(lactic-co-glycolic acid) (PLGA) 50/50 (inherent viscosity of 0.95-1.20 dl/g) from Lactel (Pelham, Ala.) [Dang, T. T. et al. Biomaterials 34 (23), 5792-5801 (2013)]. Typically, a 5 mL solution of PLGA and ibuprofen dissolved in dichloromethane, at concentrations of 40 mg/ml and 6 mg/ml respectively, was quickly added to a 25 mL solution of 1% (w/v) polyvinyl alcohol (Sigma Aldrich, St. Louis, Mo., USA) and homogenized for 60 seconds at different speeds (L5M-A, Silverson). The resulting suspension was quickly decanted into 75 mL of deionized water and stirred for 60 seconds, followed by rotary evaporation for 15 minutes. The suspension was washed three times by centrifugation at 3000 rpm for 30 seconds. The particles were collected, flash-frozen in liquid nitrogen, and lyophilized to dryness. Particle size distribution and morphology were examined under a Scanning Electron Microscopy (JSM 6390LA, JEOL). The ibuprofen loading capacity of each microparticle formulation was determined by dissolving 2 mg of particles in 1 mL of acetonitrile and comparing the resulting UV absorbance at 240 nm to a standard curve of known concentrations of ibuprofen in acetonitrile. The release kinetics from the drug-loaded subdomain was independently investigated by varying the size of ibu-PLGA particles (Table 1 and FIGS. 15 and 16). Particles of an average diameter of 14 μm and experimental drug loading of approximately 6 wt % were eventually selected for fabrication of GEL-iP to attenuate the burst release from the drug-loaded particles.

TABLE 1
Fabrication and characterization of PLGA particles with different
sizes. Average diameters were measured from SEM images.
Drug loading capacities were determined by HPLC analysis.

| Homogenizing | Average | Loading capacity |
|---|---|---|
| Speed (RPM) | diameter (μm) | (wt %) |
| 1000 | 46 ± 30.4 | 9.0 |
| 2500 | 14 ± 5.4 | 6.9 |
| 3400 | 11 ± 3.2 | 6.4 |
| 5000 | 6 ± 1.5 | 5.3 |
| 6500 | 4 ± 0.5 | 6.5 |

### 1.2 Fabrication of Particulate-Based Drug-Encapsulated Hybrid Hydrogels

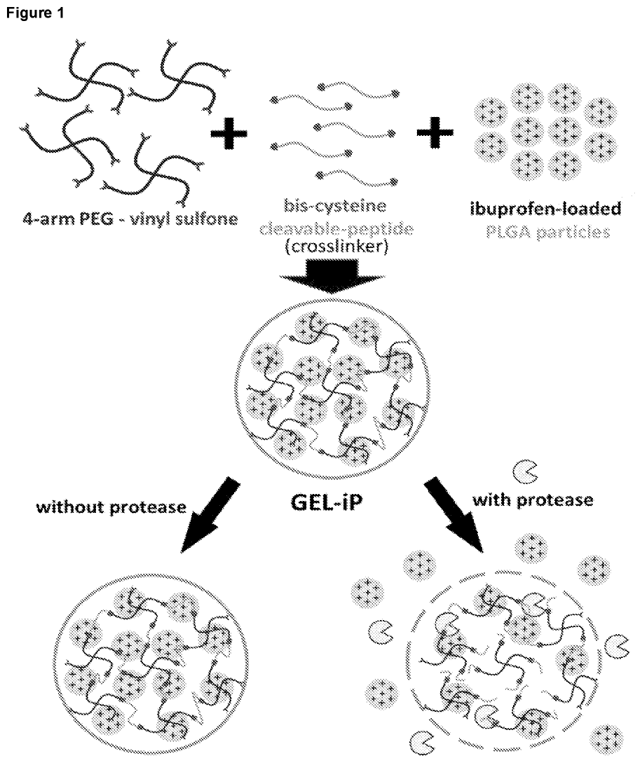

We developed a modular drug delivery platform which consisted of drug-loaded polymeric particles embedded inside a protease-cleavable hydrogel (FIG. 1). Upon exposure to protease activity, the hydrogel matrix could be proteolytically degraded to liberate the embedded particles and consequently deliver the desired therapeutic payload. Due to the modular design of this platform, the drug-loaded and protease-cleavable subdomains could be independently optimized to achieve desirable payload release. Specifically, polyethylene glycol (PEG) and poly(lactic-co-glycolic acid) (PLGA) were selected as the major polymeric components for the protease-cleavable and drug-loaded subdomains respectively due to the existing use of these polymers in clinically accepted medical products. PLGA is a synthetic polymer widely used for encapsulating therapeutic agents such as drugs and proteins due to its biodegradability and cytocompatibility [Han, F. Y. et al. Frontiers in pharmacology 7, 185-185 (2016)]. Similarly, PEG has been used as a conformal coating for immuno-protection of islets [Tomei, A. A. et al. Proceedings of the National Academy of Sciences 111 (29), 10514 (2014)] or as a component of surgical sealants [Zoia, C. et al. Journal of Applied Biomaterials & Functional Materials 13 (4), 372-375 (2015)]. In addition, since PEG is a synthetic polymer, which is available in a wide range of molecular weights with diverse multi-arm configurations and functional groups, it has been utilized in a variety of drug delivery platforms ranging from systemic, topical to injectable applications [Li, J. and Mooney, D. J. Nature Reviews Materials 1, 16071 (2016)]. Recently, the versatility of PEG-based hydrogel as a cytocompatible platform for stimuli-responsive delivery of multiple drugs and cell-based therapeutics has also been demonstrated [Badeau, B. A. Nature Chemistry 10, 251 (2018)].

Peptide-crosslinked hydrogels were prepared by reacting 4-arm poly(ethylene glycol)-vinyl sulfone (PEG-VS) (20 kDa, Sigma Aldrich, St. Louis, Mo., USA) with bis-cysteine peptides (Genscript, Hong Kong) in stoichiometric ratio. Each precursor was dissolved in either triethanolamine (TEOA) buffer (0.3M) or PBS/NaOH buffer (pH=10). Typically, to prepare 117 μL of peptide-crosslinked hydrogels with 4.2% (w/v) PEG content, 5 mg of PEG-VS was dissolved in 100 μL of a buffer solution in a glass vial and mixed with a stoichiometric amount of a peptide crosslinker dissolved in 17 μL of the same buffer solution. Hydrogels with 4.2% (w/v) PEG content were evaluated in the preliminary screening of peptide crosslinkers. Hydrogels with 1.7% (w/v) PEG content were used in all subsequent in vitro and in vivo experiments. To form hybrid hydrogels consisting of PLGA particles (with or without ibuprofen) embedded in peptide-crosslinked hydrogels, the aforementioned precursors were dissolved separately in a buffer solution containing suspended PLGA particles at a concentration of 5% (w/v). By inverting the glass vial containing the liquid mixture of PEG-VS, a peptide crosslinker, and PLGA particles at regular intervals, gelation was confirmed when this liquid mixture did not flow downward despite gravity. Examples of peptide spacer sequences, peptide substrate sequences and protease susceptibility are shown in Table 2.

TABLE 2
Potential spacers and substrates.

| Peptide Spacer | Peptide Substrate Sequences | |
|---|---|---|
| Sequences | Proteases | Corresponding Substrates |
| GRCR | HNE | APEEI↓MDRQ |
| (SEQ ID NO: 1) | | (SEQ ID NO: 14) |
| GCRG | | PMAV↓VQSVP |
| (SEQ ID NO: 2) | | (SEQ ID NO: 15) |
| GRCD | MMP-9 | GPQG↓IWGQ |
| (SEQ ID NO: 3) | | (SEQ ID NO: 13) |
| GCDR | | GPRS↓LSG |
| (SEQ ID NO: 4) | | (SEQ ID NO: 10) |
| GCDG | Cat B | GR↓RGLG |
| (SEQ ID NO: 5) | | (SEQ ID NO: 16) |
| GDCD | | DGF↓LGDD |
| (SEQ ID NO: 6) | | (SEQ ID NO: 17) |
| GCDD | | |
| (SEQ ID NO: 7) | | |

A combination of a spacer and substrate in form of SPACER-SUBSTRATE-RECAPS can be used as a crosslinker in our platform.
The cleavage site by the protease-of-interest is indicated by the “↓” symbol.

### 1.3 Screening of Peptide Crosslinkers for Optimal Gelation and Proteolytic Cleavage

In this study, the peptide crosslinkers were designed with the ultimate objectives of forming hydrogels with PEG-VS and retaining its cleavability upon exposure to MMP-9 activity. Due to the modular design of the hybrid hydrogel, the peptide crosslinker, which is a key component of the subdomain determining MMP-9 cleavability, could be independently designed. Typically, a desired peptide crosslinker consisted of an MMP-9-cleavable substrate sequence flanked by two cysteine-containing spacer sequences, each of which comprised 4 amino acids. The substrates were selected from reported peptide sequences, which had been utilized as MMP-9-sensitive components in biosensors for MMP-9 detection or as MMP-9-cleavable linkers in drug-loaded nano-carriers for chemotherapy [Biela, A. et al. Biosensors and Bioelectronics 68, 660-667 (2015); Samuelson, L. E. et al. Molecular Pharmaceutics 10 (8), 3164-3174 (2013)]. The thiol moiety on the cysteine of each terminal spacer can be deprotonated to form a thiolate [Friedman, M. et al. Journal of the American Chemical Society 87 (16), 3672-3682 (1965)] which reacts with a vinyl sulfone moiety of PEG-VS via Michael addition reaction to induce gelation [Lutolf, M. P. and Hubbell., J. A. Biomacromolecules 4 (3), 713-722 (2003)].

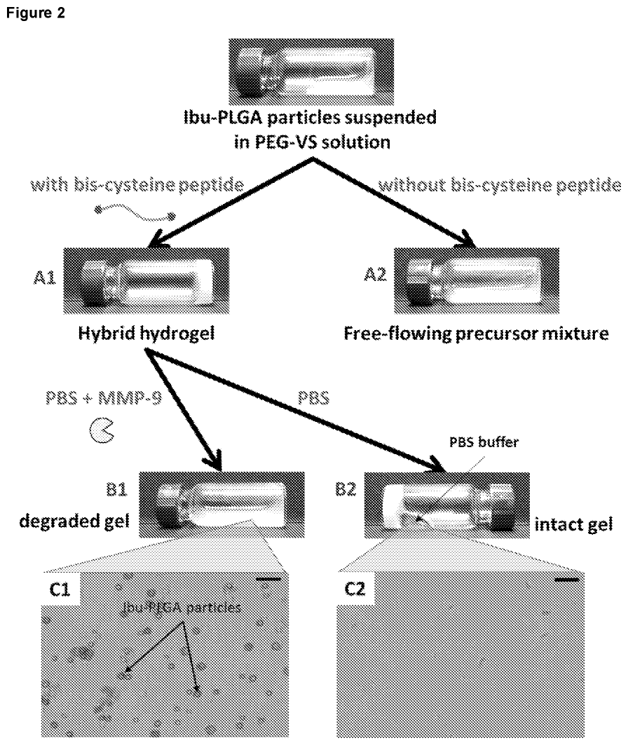

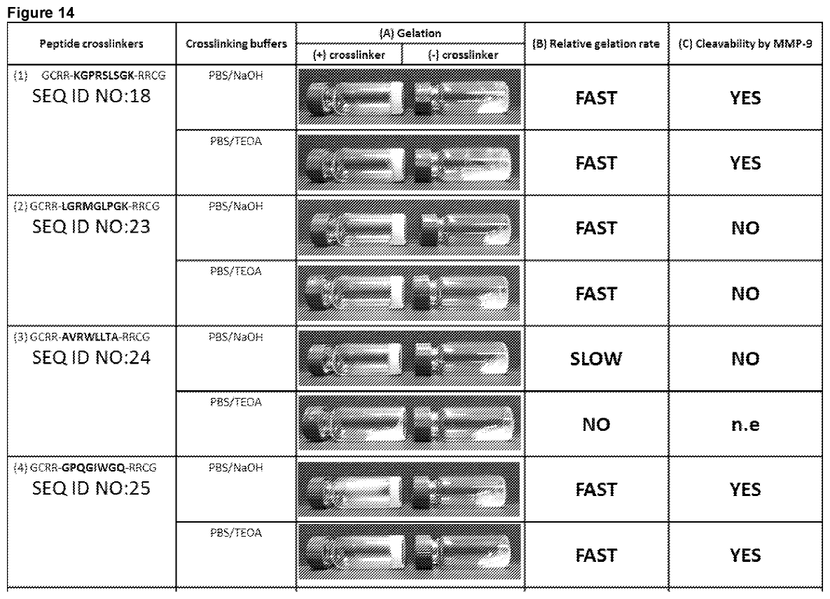

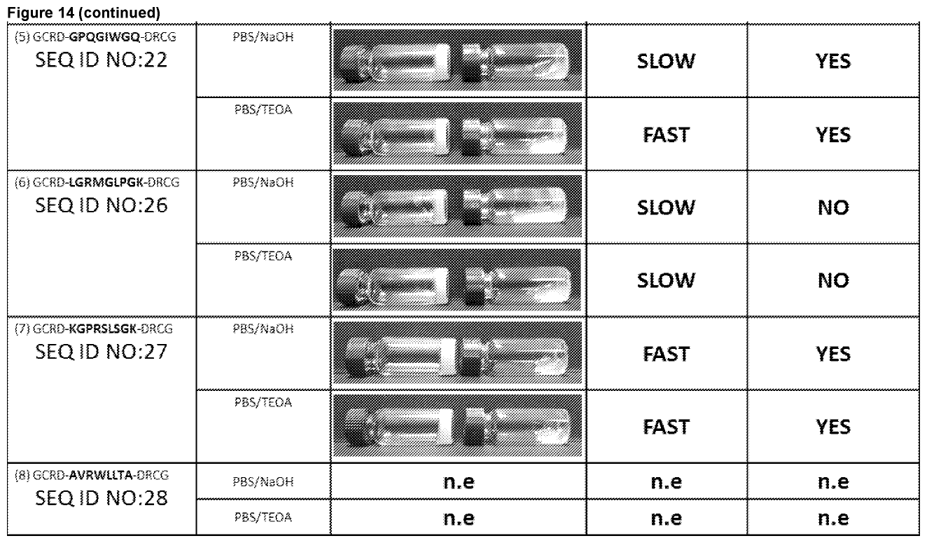

To identify an optimal peptide crosslinker for GEL-iP, we performed qualitative screening of 8 peptide sequences (Table 3 and FIG. 14) by evaluating the influence of substrate and spacer choices on the gelation and cleavability of hybrid hydrogels. In addition, buffers in which gelation occurred was also investigated because their environmental pH might affect the deprotonation of thiols and subsequently influence the crosslinking process. For each combination of substrate, spacer and buffer, the feasibility of gelation was visually inspected via a tube-inversion method. A stoichiometric amount of a peptide crosslinker was added to a glass vial containing a mixture of PEG-VS solution and suspended ibu-PLGA particles. In a parallel experiment, another vial with the same mixture composition, but without the peptide crosslinker of interest, was used as a control. The two vials were inverted at regular intervals until the mixture in one vial stopped flowing despite gravity indicating successful gelation. An example of the successful gelation of a typical hybrid hydrogel was captured in photographs of glass vials A1 and A2 (FIG. 2). In the presence of a peptide crosslinker, the white hybrid hydrogel was formed at the bottom of the glass vial A1 and did not flow downward despite gravity, confirming successful crosslinking. This solid white appearance resulted from the white ibu-PLGA particles. In the absence of any peptide crosslinker, the precursor mixture in the control vial A2 remained free-flowing, indicating absence of gelation.

TABLE 3
Examples of crosslinkers and spacers.

| Spacer | Substrate | Crosslinker sequence |
|---|---|---|
| GCRD | KGPRS↓LSGK | GCRD-KGPRS↓LSGK-DRCG |
| (SEQ ID | (SEQ ID NO: 30) | (SEQ ID NO: 27) |
| NO: 8) | LGRM↓GLPGK | GCRD-LGRM↓GLPGK-DRCG |
| | (SEQ ID NO: 11) | (SEQ ID NO: 26) |
| | AVRW↓LLTA | GCRD-AVRW↓LLTA-DRCG |
| | (SEQ ID NO: 12) | (SEQ ID NO: 28) |
| | GPQG↓IWGQ | GCRD-GPQG↓IWGQ-DRCG |
| | (SEQ ID NO: 13) | (SEQ ID NO: 22) |
| GCRR | GPQG↓IWGQ | GCRR-GPQG↓IWGQ-RRCG |
| (SEQ ID | (SEQ ID NO: 13) | (SEQ ID NO; 25) |
| NO: 9) | LGRM↓GLPGK | GCRR-LGRM↓GLPGK-RRCG |
| | (SEQ ID NO: 11) | (SEQ ID NO: 23) |
| | KGPRS↓LSGK | GCRR-KGPRS↓LSGK-RRCG |
| | (SEQ ID NO: 30) | (SEQ ID NO; 18) |
| | AVRW↓LLTA | GCRR-AVRW↓LLTA-RRCG |
| | (SEQ ID NO: 12) | (SEQ ID NO: 24) |

The cleavage site is indicated by the “↓” symbol.
The crosslinkers are in the form of SPACER-SUBSTRATE-RECAPS.

Each hybrid hydrogel of 20 μL volume in a 500 μL Eppendorf tube was incubated at 37° C. with 3 μg/ml of MMP-9 (83 kDa, Merck) in PBS buffer (DPBS/modified, without calcium and magnesium, HyClone™). In a control experiment, another hybrid hydrogel with the same composition was immersed in PBS buffer without MMP-9. After 20 hours of incubation, the medium surrounding the hybrid hydrogel was sampled onto a cover slip and observed under an optical microscope (Olympus CKX53SF, Japan) to inspect for the presence of released particles.

The selected concentration of MMP-9 is within the range of MMP-9 expression in clinical wound fluids and synovial fluids of patients with rheumatoid arthritis and osteoarthritis [Ladwig, G. P. et al. Wound Repair and Regeneration 10(1) 26-37 (2002); Li, Z. et al. Journal of Diabetes and its Complications 27(4) 380-382 (2013)]. In a typical successful cleavage by MMP-9, the photographs of vials B1 and B2 in FIG. 2 show the appearance of a hybrid hydrogel, which had the same composition as that in vial A1, after prolonged exposure to a buffer with and without MMP-9 respectively. In the presence of MMP-9, the white crosslinked hybrid hydrogel previously seen in vial A1 disappeared and only a homogenous cloudy suspension was observed in vial B1. The optical microscope image C1 verified the presence of ibu-PLGA particles in the resulting liquid mixture of vial B1 confirming the successful digestion of this hybrid hydrogel by MMP-9. In contrast, MMP-9-free PBS buffer added after gelation was observed as a clear liquid phase in vial B2. The optical microscope image C2 also confirmed the absence of any released lbu-PLGA particles in vial B2.

FIG. 14 summarizes the results from the qualitative screening of the candidate crosslinkers. The screening data in Columns (A) and (B) of FIG. 14 demonstrated that the combination of amino acids in the peptide crosslinker dictated the characteristics of the peptide sequences, which consequently affected the gelation kinetics. Most of the screened substrates resulted in successful gelation within 5 to 30 minutes. Surprisingly, only substrate AVRWLLTA (SEQ ID NO: 12), based on which peptides (3; SEQ ID NO: 24) and (8; SEQ ID NO: 28) were designed, could not offer desired reactivity of its corresponding peptide crosslinkers with PEG-VS. Specifically, peptide (3) could induce gelation by crosslinking with PEG-VS in PBS/NaOH but not in TOEA buffer. We speculate that TOEA might have acted as a surfactant to alter the conformation or arrangement of peptide (3) in aqueous solution [Jones, B. H. et al. Soft Matter 11 (18), 3572-3580 (2015)], thus impeding gelation. Interestingly, prior to addition of PEG-VS, peptide (8) self-assembled into a gel-like phase when dissolved in PBS/NaOH buffer or formed a milky solution possibly indicative of peptide self-assembly [Zhou, Q. et al. Progress in Natural Science 19 (11), 1529-1536 (2009)] in TEOA buffer. This behavior possibly hindered subsequent reaction between thiols of cysteines on this peptide and vinyl sulfones of PEG-VS thus preventing gelation.

In addition to substrate selection, spacer design (GCRR (SEQ ID NO: 9) or GCRD (SEQ ID NO: 8)) also plays an important role in the crosslinking process. For instance, in both buffers, spacer GCRR (SEQ ID NO: 9) helped peptide (2) crosslink with PEG-VS significantly faster than peptide (6) which was designed with the same substrate but a different spacer GCRD (SEQ ID NO: 8). Similarly, in PBS/NaOH, peptide (4) containing spacer GCRR (SEQ ID NO: 9) reacted with vinyl sulfones faster than peptide (5) which contained spacer GCRD (SEQ ID NO: 8). Our data was in agreement with published literature reporting that positive charges (e.g., arginine, R) close to thiol moieties of cysteines increased the crosslinking rate while negative charges (e.g., aspartic acid, D) decelerated this reaction [Lutolf, M. P. et al. Bioconjugate Chemistry 12 (6), 1051-1056 (2001)], possibly because the former stabilized the intermediate thiolates [Roos, G. et al. Antioxidants & Redox Signaling 18 (1), 94-127 (2012)].

Buffer choice can also affect the crosslinking rate because their environmental pH influences the deprotonation of thiols, leading to a change in the concentration of the intermediate thiolates [Lutolf, M. P. and Hubbell, J. A. Biomacromolecules 4 (3), 713-722 (2003)]. In addition to TEOA buffer, which is a strongly basic buffer commonly used for Michael addition but also raises cytotoxicity concern, we also investigated PBS/NaOH buffer as a potential cytocompatible alternative. As shown in FIG. 14, except sequences containing substrate AVRWLLTA (SEQ ID NO: 12), all other peptides could crosslink with PEG-VS in both buffers albeit with varying kinetics. Interestingly, with peptide (5), PBS/NaOH buffer led to a slower crosslinking kinetics than that in TEOA buffer. We speculated that the weaker basic buffer PBS/NaOH deprotonated thiols less efficiently than TOEA buffer, thus decreasing gelation rate. Our data on gelation kinetics suggested that, even for the same crosslinker design, appropriate selection of the reaction buffer is also critical in ensuring successful hydrogel formation.

Next, the results in column (C) of FIG. 14 summarizes the cleavability of each successfully crosslinked hybrid hydrogel in a solution of MMP-9. Specifically, hybrid hydrogels crosslinked with peptides (1), (4), (5) and (7) could be digested by MMP-9, as proven by the presence of released PLGA particles in the surrounding medium when the hydrogels were immersed in an MMP-9-containing buffer. Interestingly, hydrogels formed with peptides (2), (3), and (6) were not cleaved upon exposure to MMP-9, as confirmed by the equally negligible number of particles released from the hybrid hydrogels both in the presence and absence of MMP-9 (not shown). This finding was surprising, given that the substrates of these three sequences were reported to be the MMP-9-sensitive moiety in protease-activatable nano-carriers of anti-tumor drugs [Samuelson, L. E. et al. Molecular Pharmaceutics 10 (8), 3164-3174 (2013)], or electrochemical impedance sensors [Biela, A. et al. Biosensors and Bioelectronics 68, 660-667 (2015)]. A possible explanation is that the spacers which flanked both ends of the substrates might have altered the conformation of the peptide sequences, resulting in steric hindrance that prevented the protease from accessing the cleavage sites on the substrates.

To design an effective protease-triggered drug delivery platform, an optimal peptide crosslinker should induce rapid gelation and retain its cleavability in response to this protease. Among the 8 screened peptides, 3 sequences (peptides (1), (4), and (7); FIG. 14) resulted in fast gelation and successful hydrogel cleavage by MMP-9 in both buffers investigated. We selected the combination of peptide (1), which comprised 17 amino acids in the sequence GCRR-KGPRSLSGK-RRCG (SEQ ID NO: 18), and PBS/NaOH buffer to prepare the desired hybrid hydrogel GEL-iP for subsequent investigation.

### 1.4 In Vitro Study of Drug Release Kinetics from Particulate-Based Drug-Encapsulated Hybrid Hydrogels

Two types of hybrid hydrogels were compared in this in vitro release study. GEL-iP was a hydrogel embedded with ibuprofen-loaded PLGA particles (ibu-PLGA particles) crosslinked with cleavable peptide (1) GCRR-KGPRSLSGK-RRCG (SEQ ID NO: 18). ScrGEL-iP was a hydrogel embedded with ibu-PLGA particles and crosslinked with non-cleavable scrambled peptide GCRR-KSSRGGPLK-RRCG (SEQ ID NO: 29). Briefly, 40 μL of GEL-iP was immersed in 500 μL of a PBS solution with or without 3 μg/ml of MMP-9 in a 1.5 mL tube while 40 μL of scrGEL-iP was only exposed to the MMP-9-containing PBS solution. In a control experiment with GEL-iP, MMP-9 inhibitor I (Merck) was added along with MMP-9. Each tube was maintained at a temperature of 37° C. and a shaking speed of 30 rpm on a Multi Bio RS-24 rotator (BioSan). At predetermined intervals, 10 μL aliquot of the liquid mixture from each tube was collected and added to 90 μL of acetonitrile. The resulting sample was passed through a 0.22 μm syringe filter and stored at 4° C. A 10 μL volume of fresh PBS solution with or without 3 μg/ml of MMP-9 was added to each tube to replace the aliquoted volume. After 24 hours, each hybrid hydrogel together with its remaining liquid mixture was completely dissolved in acetonitrile. The concentration of ibuprofen in all collected samples was quantified by RP-HPLC. The percentage of drug release at each time point was calculated by normalizing the cumulative amount of drug collected at each point with the initial amount of drug in each tube [Dang, T. T. et al. Biomaterials 32 (19), 4464-4470 (2011)]. The release kinetics reported for each hybrid hydrogel was obtained from the average of quadruplicate experiments.

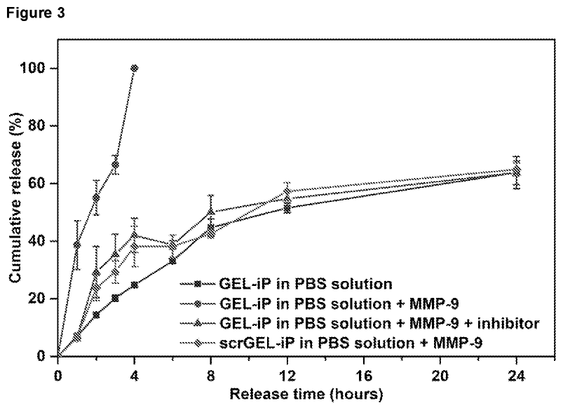

As shown in FIG. 3, exposing GEL-iP to MMP-9 for 4 hours significantly boosted the cumulative drug release to 100% compared to only 25% in the absence of MMP-9. When an MMP-9 inhibitor was added together with MMP-9, the amount of released ibuprofen was significantly suppressed. This was because the proteolytic activity of MMP-9 could be selectively blocked by the small molecule inhibitor. Hence, MMP-9 could not cleave the peptide crosslinker to break down the hydrogel matrix, preventing the triggered release of ibuprofen. In addition, while GEL-iP was completely digested to release 100% of ibuprofen in the presence of MMP-9 within the first 4 hours, only 40% was released from the hybrid hydrogel crosslinked by the scrambled peptide (scrGEL-iP) at the same condition. In the presence of MMP-9, the higher amount of ibuprofen delivered from GEL-iP compared to that from scrGEL-iP confirmed the role of the cleavable peptide (1) in enabling the MMP-9-induced degradation of GEL-iP to trigger increased drug release. Overall, the data in FIG. 3 proved that the triggered release was due to the cleavage activity of MMP-9 on its associated peptide substrate.

### Evaluating In Vitro Inhibitory Effects of Particulate-Based Drug-Encapsulated Hybrid Hydrogels on Macrophages

The drug released from GEL-iP upon exposure to MMP-9 trigger was evaluated by investigating its in vitro inhibitory effects on the proliferation of RAW 264.7 murine macrophages. Published studies previously demonstrated that local macrophage proliferation, rather than monocyte recruitment, dominates lesional accumulation of this cell type in inflammation-associated diseases such as atherosclerosis and obesity-associated adipose tissue inflammation [Amano, S. U. et al. (2014)]. Therefore, macrophage self-division has been postulated as a potential target for therapeutic modulation of inflammation.

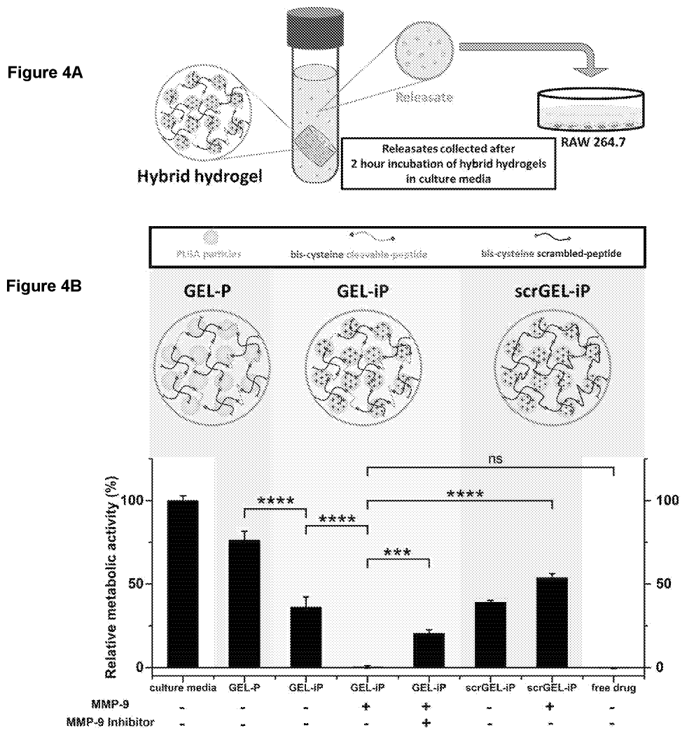

RAW 264.7 murine macrophages were cultured in high glucose DMEM (Gibco Laboratories) supplemented with 10% FBS (Gibco Laboratories), and 1% penicillin/streptomycin (Gibco Laboratories) at 37° C. in 5% CO2 atmosphere. RAW 264.7 macrophages at passages of 20-30 were seeded in 96-well plates (Corning®) at an initial seeding density of 2×10⁴ cells/well and incubated at 37° C. for 24 hours. A volume of 100 μL of each hybrid hydrogel (GEL-iP and scrGEL-iP) fabricated with 1.7% (w/v) of PEG and 5% (w/v) of ibuprofen-loaded PLGA microparticles was incubated in 500 μL of phenol red-free DMEM (Gibco Laboratories) culture medium for 2 hours in the presence and absence of 3 μg/ml of MMP-9 (FIG. 4A). MMP-9-cleavable hydrogel embedded with ibuprofen-free PLGA particles (GEL-P) was also included as a control hybrid hydrogel. In one group of GEL-iP samples, MMP-9 inhibitor I was added along with MMP-9 during incubation. Following the incubation, 200 μl of the culture medium was collected from each hybrid hydrogel formulation as releasate to treat seeded RAW 264.7 macrophages for 72 hours. A volume of 200 μL of fresh culture medium and 0.6 mg/mL of freely dissolved ibuprofen were also used to treat macrophages as negative and positive controls respectively. This dosage of freely dissolved ibuprofen was normalized based on the amount of drug loaded in each hybrid hydrogel. Afterwards, the releasates were removed from the treated macrophages; and the in vitro metabolic activity of treated cells was evaluated using WST-1 cell proliferation assay (Abcam) as per manufacturer's protocol. Specifically, 200 μL of culture medium containing WST-1 reagent at a 10:1 volume ratio was added into each well and incubated at 37° C. for 3 hours. A volume of 100 μL of this medium was then transferred into a new 96-well plate; and its absorbance at 450 nm and 690 nm was recorded using a microplate reader (SpectraMax M5). To calculate relative metabolic activity, all the absorbance values at 450 nm were subtracted by the values at the reference wavelength of 690 nm and the background absorbance to achieve corrected absorbance values. Afterward, the relative metabolic activity was calculated as follows:

Relative metabolic activity=ATest/AControl*100%

Where ATest and AControl were the corrected absorbance values of solutions collected from the cells which were treated with the releasates and the fresh culture medium respectively.

As shown in FIG. 4B, the releasate obtained by MMP-9 digestion of GEL-iP completely inhibited macrophage proliferation (˜0%) while that obtained from GEL-iP in the absence of MMP-9 resulted in a higher metabolic activity (˜40%) from the treated cells. This data indicated that GEL-iP released a higher amount of ibuprofen to further reduce macrophage proliferation when triggered by an MMP-9 concentration of 3 μg/ml, which simulated upregulated protease expression due to increased inflammation in chronic diseases [Ladwig, G. P. et al. Wound Repair and Regeneration 10(1) 26-37 (2002); Li, Z. et al. (2013)]. Furthermore, in the absence of MMP-9, the metabolic activity of the macrophages treated with the releasate obtained from GEL-iP (˜40%) was significantly higher than that of the cells treated with an equivalent dosage of the freely dissolved drug (˜0%), which mimicked uncontrolled drug delivery by systemic administration. Thus, in MMP-9 absence which simulated non-inflammatory physiological conditions [Roomi, M. W. et al. (2009)], GEL-iP was able to release less ibuprofen than the freely dissolved drug to subsequently minimize the effect of the drug on macrophages. This feature of Gel-iP suggested its potential to mitigate drug-induced side effects in non-inflammatory conditions by decreasing excessive drug dosage resulted from uncontrolled release kinetics during systemic drug administration [Youssef, J. et al. Rheumatic diseases clinics of North America 42(1) 157-176 (2016)].

In addition, when MMP-9 inhibitor was added along with MMP-9, the metabolic activity of macrophages was restored to 20% from the complete inhibition (˜0%) observed in the absence of this inhibitor, proving that active MMP-9 is essential to achieve the desired inhibitory effects of releasates on macrophages. In the presence of MMP-9, while ibuprofen released from GEL-iP could completely inhibit macrophage proliferation, this activity still remained at 55% when the cells were treated with the releasate from non-cleavable scrGEL-iP. The findings from the control experiments with MMP-9 inhibitor and scrGEL-iP established the critical role of both active MMP-9 and its associated cleavable peptide in triggering ibuprofen release from Gel-iP to modulate macrophage proliferation. Overall, GEL-iP is a promising drug delivery platform which can be triggered by protease activity to release anti-inflammatory drugs and potentially modulate activity of immune cells.

While our main objective was to develop a delivery platform that releases drug only when triggered by protease activity with minimal release in the absence of this stimulus, some concerns arguably remain regarding the basal amount of ibuprofen released from GEL-iP in the absence of MMP-9. This may be due to passive drug diffusion from the surface of ibu-PLGA microparticles and accounted for the partial inhibition (˜40%) of macrophages treated with the releasate from Gel-iP in the absence of MMP-9 and its inhibitor. Nonetheless, in most actual clinical applications that require administration of an anti-inflammatory drug, some level of inflammation exists. Thus, this basal drug release can be useful for management of low level of inflammation and associated symptoms such as pain and swelling [Steinmeyer, J. (2000)] to minimize exacerbation of the inflammatory response [Sutherland, E. R. et al. (2003)]. If the inflammation suddenly worsens with a resultant increase in MMP-9 activity as in the case of an arthritic flare or infection of chronic wounds, GEL-iP will be triggered to release more ibuprofen to cope with the increased severity of inflammation.

### Example 2: In Vivo Evaluation of Protease-Responsive Particulate-Based Drug-Encapsulated Hybrid Hydrogels

### 2.1 Animal Care of Immuno-Competent SKH-1 Mice

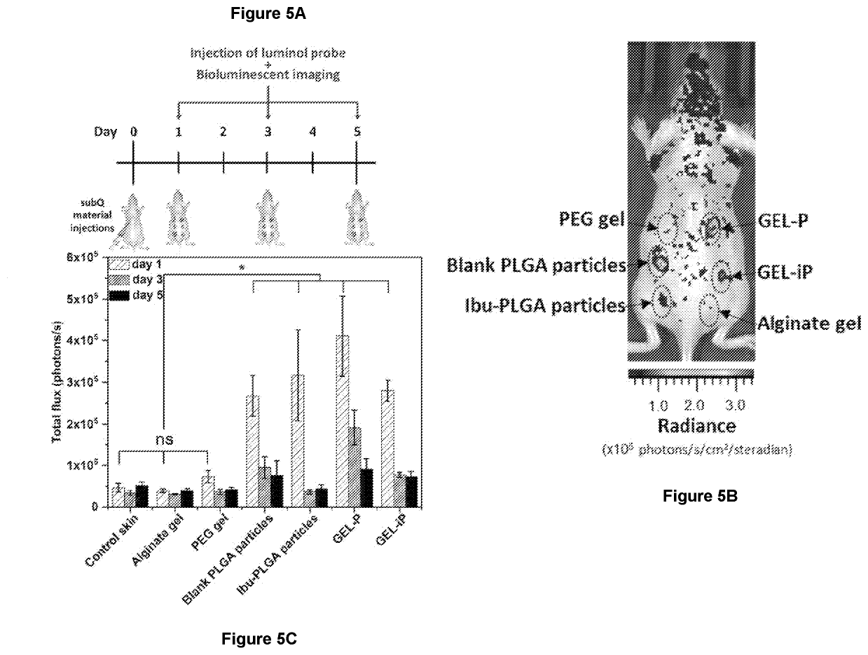

To further evaluate the potential use of GEL-iP as an injectable drug delivery platform for subcutaneous applications, we assessed the immuno-compatibility of GEL-iP and its constituent materials in vivo. An immunocompetent mouse model SKH-1E was utilized to investigate the effect of these materials on subcutaneous host response for up to 5 days (FIG. 5). This study was conducted following an animal protocol (protocol number A0343) approved by the Institutional Animal Care and Use Committee (IACUC) of Nanyang Technological University (NTU), Singapore. All animal experiments followed the National Advisory Committee for the Laboratory Animal Research (NACLAR), which complies with the National Institutes of Health guide for the care and use of laboratory animals (NIH Publications No. 8023, revised in 1978). Female SKH-1E mice (F1) at the age of 10 weeks were bred in-house from breeder mice purchased from Charles River Laboratories (Wilmington, Mass., USA). The mice were housed under standard conditions with a 12-hour light/dark cycle at the animal facilities of Lee Kong Chian School of Medicine, NTU. Both water and food were provided ad libitum.

### 2.2 Subcutaneous Injection of Polymeric Microparticles

Before subcutaneous injection of materials, mice were kept under inhaled anesthesia using 3% isoflurane in oxygen. Six different material formulations were subcutaneously injected in an array format on the dorsal side of each mouse. Specifically, a volume of 50 μL of PBS buffer containing ibu-PLGA particles (50 mg/ml), ibuprofen-free blank PLGA particles (50 mg/ml), or 1% (w/v) alginate hydrogel (PRONOVA™ SLG20, FMC BioPolymer) was injected. For each hydrogel formulation such as GEL-iP, GEL-P, or PEG hydrogel crosslinked by peptide (1) (GCRR-KGPRSLSGK-RRCG; SEQ ID NO: 18) without PLGA particles (PEG gel), 50 μL of a solution containing the corresponding precursors was injected. For example, in situ formation of GEL-iP was induced by subcutaneously injecting 50 μL of a PBS/NaOH buffer containing PEG-VS, the peptide crosslinker, and ibu-PLGA particles on the dorsal side of the mouse.

### 2.3 Non-Invasive Bioluminescent Imaging of SKH-1E Mice

ROS activity was quantified using luminol which was oxidized by ROS to emit bioluminescent signal as reported in other studies [Liu, W. F. et al. Biomaterials 32 (7), 1796-1801 (2011)]. Briefly, prior to imaging, the mice were injected with 5 mg of sodium luminol (Sigma Aldrich, St. Louis, Mo., USA) dissolved in 100 μL PBS into the peritoneum. Twenty minutes after this injection, the mice were imaged using the IVIS Spectrum CT system (Caliper Life Sciences) with 180 s exposure. Total flux (photons/s) was determined over a region of interest (ROI) (cm²) around the injection site using Living Image 3.1 software.

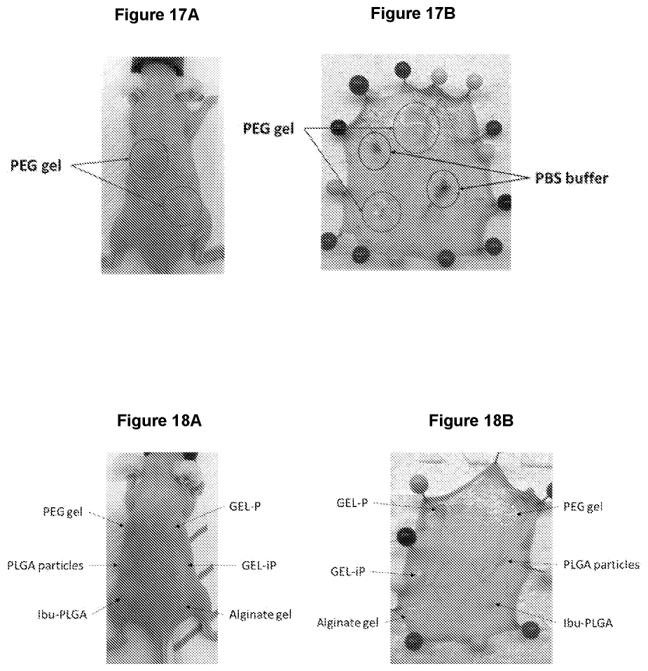

In a separate preliminary experiment, in situ gelation was confirmed by the presence of crosslinked hydrogels at the subcutaneous space of the excised skin 15 minutes post-injection (FIG. 17). To evaluate the effect of constituent materials of GEL-iP, ibu-PLGA particles and ibuprofen-free PLGA particles were also investigated. Furthermore, calcium-crosslinked alginate hydrogel (alginate gel) was used as a negative control because its subcutaneous immuno-compatibility in mice was previously reported [Liu, W. F. et al. Biomaterials 32 (7), 1796-1801 (2011)].

Multiple in vitro and in vivo studies have quantified reactive oxygen species (ROS) activity generated by activated phagocytes to characterize material-induced host response [Dang, T. T. et al. Biomaterials 34 (23), 5792-5801 (2013); Dang, T. T. et al. Biomaterials 2011, 32 (19), 4464-4470 (2011)]. In this experiment, we used a non-invasive imaging technique to quantify bioluminescent signals emitted due to the oxidation of luminol imaging probe by ROS at the material injection site on days 1, 3, and 5 post-injection (FIG. 5A) [Dang, T. T. et al. Biomaterials 34 (23), 5792-5801 (2013); Dang, T. T. et al. Biomaterials 32 (19), 4464-4470 (2011); Liu, W. F. et al. Biomaterials 32 (7), 1796-1801 (2011)]. FIG. 5B shows a bioluminescent image of a representative mouse on day 3 while FIG. 5C presents the quantified ROS activity induced by different material formulations over a period of 5 days. On day 1, peptide-crosslinked PEG gel without PLGA particles induced a comparable ROS activity to that of alginate gel, confirming the immuno-compatibility of PEG gel in the subcutaneous space. This data also verified that choosing PBS/NaOH as the buffer for precursor solution in the preparation of PEG gel did not induce adverse effect on ROS production by immune cells. Moreover, on day 5, the disappearance of the crosslinked PEG gel at the subcutaneous space of the excised skin suggested that the PEG gel had completely degraded (FIG. 18). Interestingly, all PLGA-containing formulations resulted in a higher level of ROS production than that caused by PEG gel and alginate gel, both of which did not contain PLGA. Even though PLGA has been approved by FDA for use in several drug delivery applications [Han, F. Y. et al. Frontiers in pharmacology 7, 185-185 (2016)], its hydrophobicity possibly led to an acute inflammation [Seong, S.-Y. and Matzinger, P. (2004)] and an associated increase in ROS activity [Dang, T. T. et al. Biomaterials 34 (23), 5792-5801 (2013)] as observed in this study. However, this PLGA-associated ROS activity on day 1 eventually reduced to the same background level as that of the control skin on day 5, suggesting that this PLGA-induced elevation of ROS activity was transient. Thus, PLGA could still be considered immuno-compatible in the subcutaneous space of SKH-1E mice. Overall, our findings suggested that constituent materials of GEL-iP, namely PEG hydrogels and PLGA particles, were appropriate immuno-compatible materials for the design of the hybrid hydrogel. In addition, our bio-orthogonal chemical gelation strategy and buffer choice did not induce adverse increase in ROS activity.

### Example 3: Particulate-Based Protease-Cleavable Hydrogels with Plural Responsivity

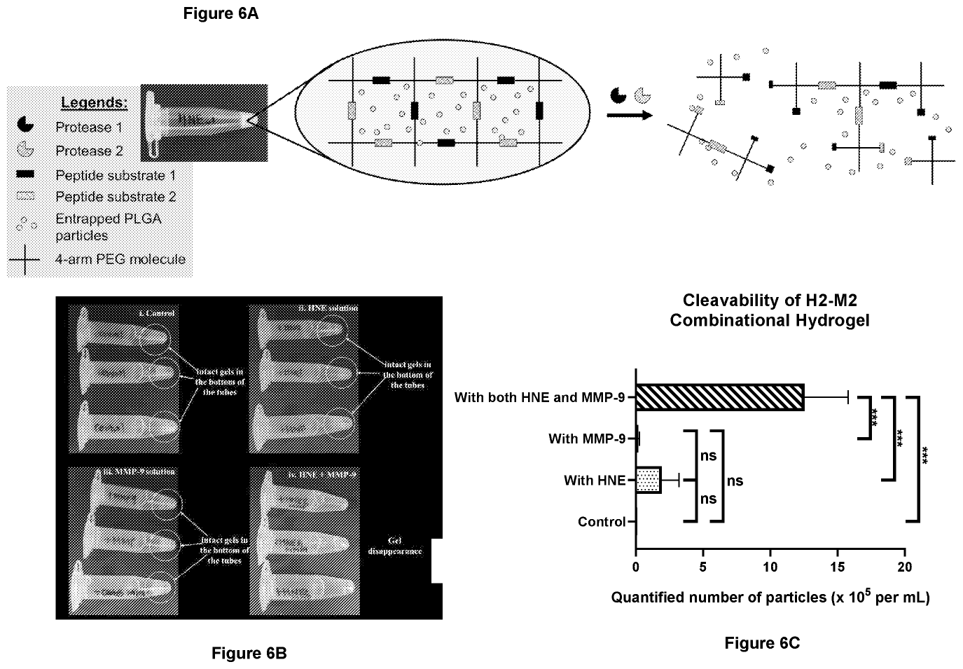

We demonstrated that particulate-based protease-cleavable hydrogels with plural responsivity were digested the fastest upon the coexistence of more than one disease-specific proteases, enhancing the specificity to target inflammatory diseases. Typically, the H2-M2 combinational hydrogels, which were crosslinked by a combination of a HNE peptide substrate (H2) and MMP-9 peptide substrate (M2) (Table 4), remained intact with the exposure to only a single protease, either HNE or MMP-9. However, the hydrogels were fully degraded when both proteases were added (FIG. 6A). One-way ANOVA statistical analysis conducted on the quantified average number of PLGA particles in the supernatant also showed that the number of particles released in the presence of both proteases is significantly greater (p 0.001) when compared against hydrogels with single protease and the control (Figure. 6B). Additionally, the results also indicated that H2 and M2 peptide substrates were preferentially cleaved by HNE and MMP-9 proteases respectively.

TABLE 4
Peptide substrates with
protease cleavability and specificity

| | Abbre- | Peptide substrate | SEQ |
|---|---|---|---|
| Protease | viation | sequence | ID NO |
| HNE | H1 | GRCR-APEEI↓MDRQ-RCRG | 35 |
| | H2 | GRCR-PMAV↓VQSVP-RCRG | 31 |
| MMP-9 | M1 | GRCR-GPQG↓IWGQ-RCRG | 36 |
| | M2 | GRCR-GPRS↓LSG-RCRG | 32 |
| Cat B | C1 | GRCR-GR↓RGLG-RCRG | 37 |
| | C2 | GRCR-DGF↓LGDD-RCRG | 38 |

### Example 4: Fabrication of Composite Dressings Comprising Protease-Responsive Particulate-Based Drug-Encapsulated Hybrid Hydrogels

### 3.1 In Vitro Study of Drug Release Kinetics from Composite Dressings

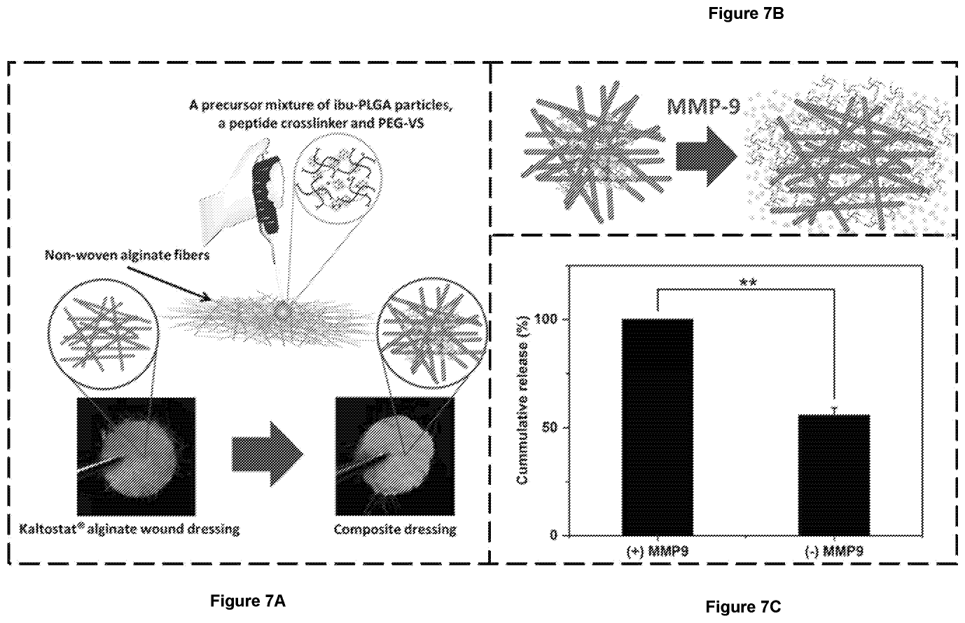

To illustrate the versatility of this drug delivery platform for topical applications, we incorporated the hybrid hydrogel GEL-iP with Kaltostat® wound dressing to form a composite dressing (FIG. 7A). The ultimate purpose of this composite dressing is to release ibuprofen upon exposure to elevated MMP-9 levels in the exudate of chronic wounds [Ladwig, G. P. et al. Wound Repair and Regeneration 10(1) 26-37 (2002); Li, Z. et al. Journal of Diabetes and its Complications 27(4) 380-382 (2013)] (FIG. 7B) for inflammation and pain management. To fabricate a composite dressing, peptide (1) (GCRR-KGPRSLSGK-RRCG; SEQ ID NO: 18) and PEG-VS were dissolved separately in PBS/NaOH buffer containing suspended ibu-PLGA particles at a concentration of 5% (w/v). These two precursors were mixed together before 20 μL of the precursor mixture was quickly deposited onto a circular sheet of Kaltostat® alginate wound dressing with a diameter of 6 mm (FIG. 7A). After gelation, the composite dressing was flash-frozen in liquid nitrogen and lyophilized to dryness. Two composite dressings were compared in this in vitro release study. Briefly, a composite dressing containing 20 μL of GEL-iP was immersed in 500 μL of a PBS solution with 3 μg/ml of MMP-9 in a 1.5 mL tube while another dressing was only exposed to the MMP-9-free PBS solution.

Since the newly-formed composite dressing was saturated with the water from the precursor mixture, we observed that its ability to further absorb liquid significantly decreased. Thus, the composite dressing was lyophilized to restore its absorbability. This dressing was then investigated for its ability to release ibuprofen upon exposure to MMP-9 by immersing it in a buffer solution with or without MMP-9 for 24 hours. After 24-hour incubation, the composite dressing rapidly absorbed the buffer and released nearly 100% of loaded ibuprofen in the presence of MMP-9 compared to only 56% in the absence of MMP-9 (FIG. 7C). Overall, the hybrid hydrogel GEL-iP provided a versatile triggered drug release platform which was potentially suitable for not only injectable formulations (FIG. 5) but also topical applications (FIG. 7).

### Example 5: Modular Conjugate-Based Hydrogel with Singular or Plural Protease Responsivity

### 5.1 Conjugation of Drug to Protease-Sensitive Peptide Anchor

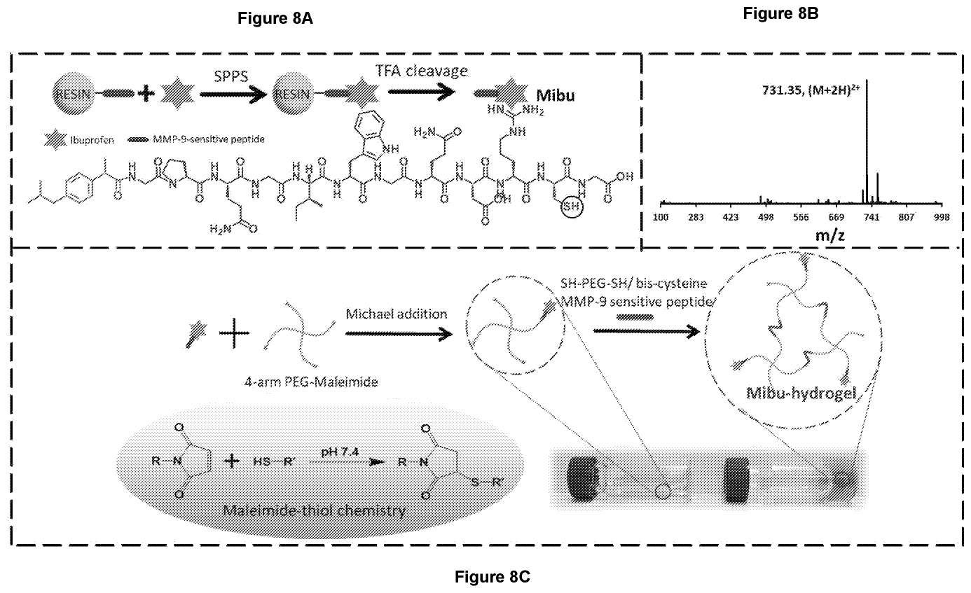

We have developed a robust process to synthesize and purify the new ibuprofen-peptide conjugate. The process flowchart is shown in FIG. 8A. The method is briefly as follows.

First, ibuprofen, a non-steroid anti-inflammatory drug (NSAID), was conjugated to the N-terminus of a peptide sequence, GPQGIWGQ-DRCG (SEQ ID NO: 19), to form ibu-GPQGIWGQ-DRCG (SEQ ID NO: 19) using solid phase peptide synthesis (SPPS). Fmoc-protected peptide was first synthesized on 0.3 mmol/g scale on Rink amide resin using standard manual solid phase peptide synthesis. Prior to ibuprofen conjugation, the Fmoc protection group was removed with 20% piperidine. An amount of 50 mg of the Fmoc-free resin was then dispersed in 500 μL of DMF along with 9.28 mg of ibuprofen. The reaction occurred when 34.2 μL of 1M PyBOP solution in DMF and 6 μL of DIPEA were added into the resin dispersion. After 18 hours, the resin was washed with DMF then dichloromethane (DCM) several times. Next, ibu-GPQGIWGQ-DRCG (SEQ ID NO: 19) was cleaved off the resin using a cleavage cocktail containing 95% trifluoroacetic acid, 2.5% water and 2.5% triisopropylsilane (TIPS) for 60 min at room temperature. The product was precipitated in cold ether, then kept under vacuum to dryness. The identity of the peptide-drug conjugation was confirmed with MS, MS m/z: 731.35 [M+2H]2+. The LC-MS data indicated that the drug has been successfully conjugated to the N-terminus of the peptide sequence, GPQGIWGQ-DRCG (SEQ ID NO: 19), as the observed molecular weight of ibu-GPQGIWGQ-DRCG (SEQ ID NO: 19) matched the theoretically predicted values from ChemDraw® (FIG. 8B).

Second, ibu-GPQGIWGQ-DRCG (SEQ ID NO: 19) was linked to the hydrogel using the following procedure. 4-arm poly(ethylene glycol)-maleimide (4-PEG-Mal) (20 kDa, Sigma Aldrich, St. Louis, Mo., USA) first reacted to ibu-GPQGIWGQ-DRCG (SEQ ID NO: 19) with a mole ratio of 1/1. Bis-cysteine peptides (Genscript, Hong Kong), in stoichiometric ratio to 4-PEG-Mal (albeit the amount of maleimide groups occupied by ibu-GPQGIWGQ-DRCG (SEQ ID NO: 19)) was then added to the reaction mixture. Each precursor was dissolved in PBS buffer. Typically, to prepare 117 μL of peptide-crosslinked hydrogels with 4.2% (w/v) PEG content, 5 mg of 4-PEG-Mal was dissolved in 100 μL of PBS along with 0.40 mg of ibu-GPQGIWGQ-DRCG (SEQ ID NO: 19) in a glass vial. Next, a stoichiometric amount of a peptide crosslinker dissolved in 17 μL of PBS was added to the solution.

As demonstrated by the schematics in FIG. 8C, addition of a bis-cysteine MMP9-sensitive peptide crosslinker (GCRDGPQGIWGQDRCG; SEQ ID NO: 22) to a buffered mixture of 4-arm PEG-maleimide and ibu-GPQGIWGQ-DRCG (SEQ ID NO: 19) resulted in the formation of a crosslinked hydrogel which did not flow downward despite gravity.

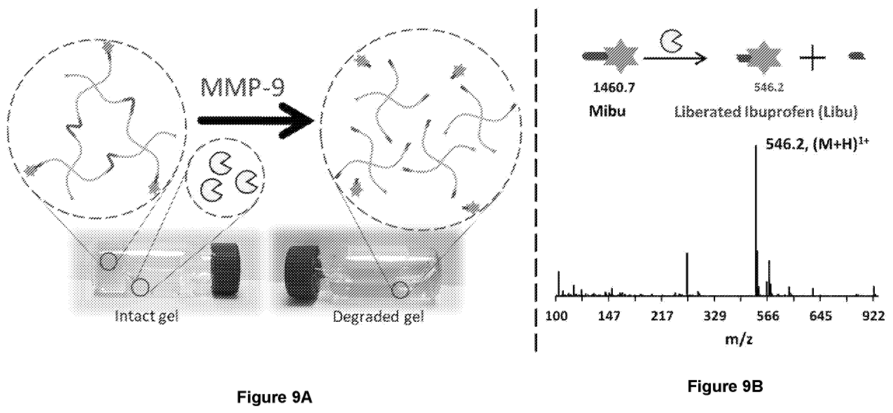

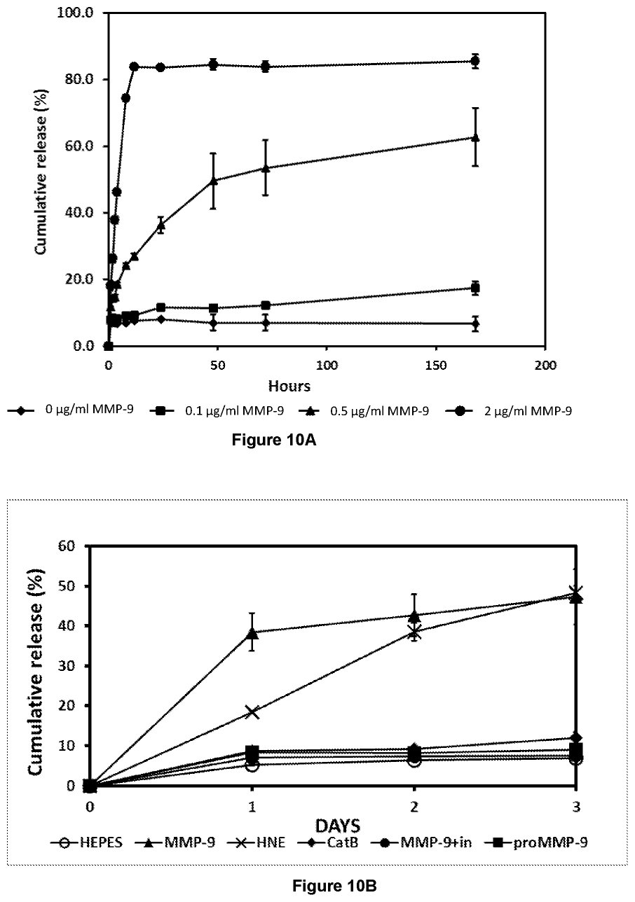

FIG. 9 supports the hypothesis that protease-sensitive drug release can be achieved from an ibuprofen-conjugated PEG hydrogel upon exposure to clinically relevant protease concentration of 1 ug/ml. FIG. 9A shows complete digestion of the crosslinked hydrogel after 3 days. LC-MS analysis confirmed the presence of the liberated ibuprofen bearing fragment (ibu-GPQG, LC peak at elution time of 14.6 min, MS peak at m/z=546.20) in the supernatant collected after 2 days of hydrogel exposure to MMP9 solution (FIG. 9B). In contrast, this peak is absent from the supernatant collected when the hydrogel was exposed to a control buffer without MMP9. Since the concentration of active MMP9 in human wound fluid falls within the range of 0.3-4.8 μg/ml [29-31], our data in FIG. 10A support the feasibility of drug release at a clinically relevant protease concentration. Furthermore, the peptide sequence, GPRSLSGRRCG (SEQ ID NO: 20), as a spacer illustrated a good specificity toward MMP-9 compared to cathepsin B and human neutrophil elastase (HNE), as shown in FIG. 10B.

Third, preparation of plural protease-cleavable hydrogels was accomplished through Michael-type addition reaction of thiol-containing peptides onto 4-PEG-Mal. A gel of 117 μL volume containing 4.2% (w/v) 4-PEG-Mal was formed by dissolving 5 mg 4-PEG-Mal in 100 μL PBS buffer and reacting this solution with 17 μL of a combination of peptide sequences, such as 0.005 mg of GRCR-PMAVVQSVP-RCRG (SEQ ID NO: 31) and 0.0045 mg of GRCR-GPRSLSG-RCRG (SEQ ID NO: 32).

### 5.2 Tunable In Vitro Drug Release from Conjugate-Based Hydrogel

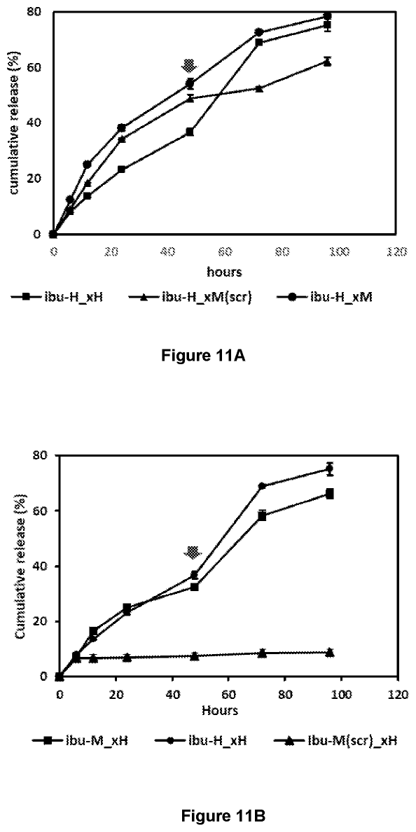

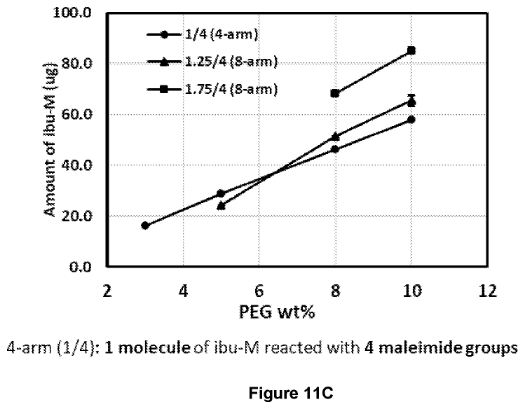

Quantitative data determined from high performance liquid chromatography (HPLC) as shown in FIG. 11A-B suggests that, by varying the choice of bis-cysteine crosslinkers or anchors, the tunable drug release from the drug-conjugated hydrogel might be achieved. Specifically, in FIG. 11A, when the same anchor H was used, cumulative drug release was decreased when the crosslinker was changed from peptide xM (GCRR-GPRSLSG-RRCG, SEQ ID 21) to peptide xH (GCRD-GPQGIWGQ-DRCG, SEQ ID 22). In addition, in FIG. 11B, when the same crosslinker xH (GCRD-GPQGIWGQ-DRCG, SEQ ID 22) was used, the cumulative drug release was decreased when the anchor was changed from peptide H (GPQGIWGQ-DRCG, SEQ ID 19) to peptide M (GPRSLSG-RRCG, SEQ ID 20). As shown in FIG. 11C, increasing the weight ratio of the hydrogel from 3 to 10 w/v % increased the loading capacity confirming the tunability of total loaded dosage. In addition, 8-arm-PEG-Maleimide could load more ibuprofen than 4-arm-PEG-Maleimide did.

### 5.3 Inflammation-Induced In Vivo Drug Release from Conjugated-Based Hydrogel

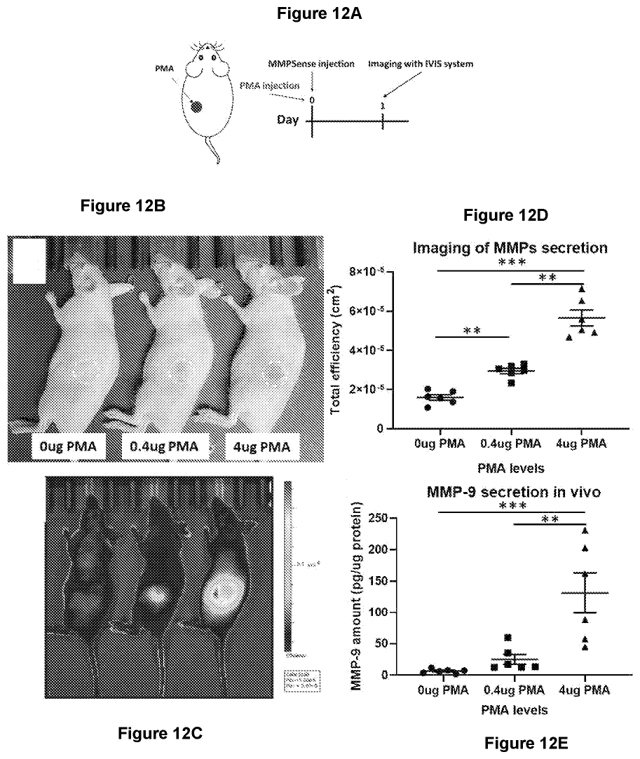

We also obtained preliminary data suggesting the drug release from our drug-conjugated platform was able to release higher drug dose in response to increased inflammation in vivo. We established a mouse model of skin inflammation using phorbol 12-myristate 13-acetate (PMA) as a stimulant (FIG. 12A) which could induce the upregulation of MMPs captured by MMPSense probe. At the PMA injection sites 24 hours post PMA injection, we observed a red area whose extent was associated with the increase in PMA amounts injected (FIG. 12B). Specifically, 4 ug of PMA induced a larger and bolder red area compared to that induced by 0.4 μg of PMA, while PMA-free PBS buffer did not cause any change on the skin appearance. The MMP activity was monitored after 24 hours of PMA injection and the quantification of fluorescent signals showed a significantly higher MMP activity induced by 4 μg of PMA compared to that caused by 0.4 μg of PMA, with 1.9-fold difference (FIG. 12C, D). In addition, there was a basal of amount of MMP which generated the lowest fluorescent signal at PMA-free PBS buffer injection site. Thus, as more PMA was subcutaneously injected, the fluorescent intensity rose; and more importantly this increase in signal intensity correlated with the extent of the red area on the dorsal side of mice. Protein expression was also studied as skin tissue at the PMA injection sites were retrieved for MMP-9 quantification using ELISA kit (FIG. 12E). A trend of MMP-9 upregulation was observed to correlate with the varying levels of inflammatory severity induced by different concentrations of PMA injected. Specifically, 4 μg of PMA induced a significantly higher amount of MMP-9 secreted than that caused by 0.4 μg and 0 μg PMA, with 5 and 10-fold difference, respectively. Overall, PMA subcutaneous injection with increasing amounts could induce subcutaneous inflammation with increasing levels of severity, resulting in more MMP-9 secretion.

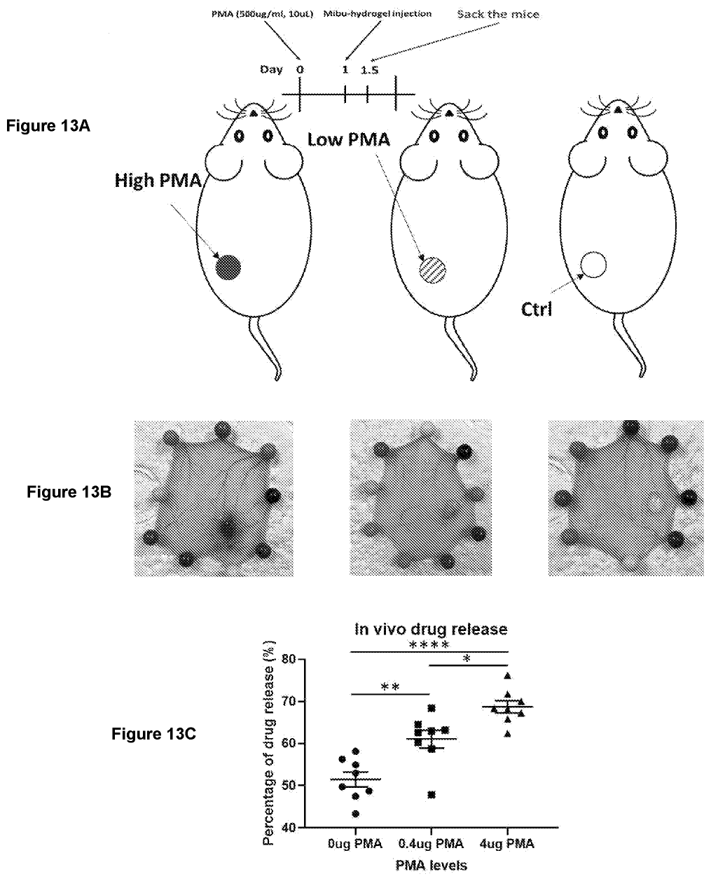

To investigate the in vivo inflammation-triggered release of drug from the ibuprofen-conjugated PEG hydrogels, ibu-M_xM hydrogel was formed in situ by injecting its precursor solution in the subcutaneous space of the inflamed sites on the dorsal side of SKH-1E mice (FIG. 13A). Either one of three levels of inflammation which was created one day in advance was used to trigger the drug release, and the percentage of drug release was then calculated based on the remaining amount of ibu-Mf inside the gel blobs 12 hours post hydrogel injection. FIG. 13B shows the colorless and transparent appearance of hydrogels at PMA-free injection sites in contrast with the yellow appearance with unclear boundaries at the sites exposed to 4 ug PMA. FIG. 13C shows 70% of ibu-Mf was released from the gels exposed to 4 μg PMA compared to 60% and 45% of drug released from those exposed to 0.4 μg PMA and PMA-free PBS buffer, respectively. Thus, ibu-Mf release was observed to positively correlate with the varying levels of inflammatory severity, proving its in vivo protease-triggered release mechanism. Furthermore, 45% of drug released in the absence of PMA could possibly due to the injection procedure during PMA and hydrogel administration which induced a mild inflammation associated with upregulated MMP-9 secretion, resulting in the release of ibu-Mf. Thus, this hydrogel configuration was sensitive to inflammation even with minor severity. The protease-sensitivity of the hydrogel could potentially be adjusted by changing the peptide anchor and/or crosslinker components. Overall, the ability of drug-conjugated PEG hydrogels to release more drug under more severe inflammation suggested its potential to cope with different levels of inflammatory severity.

### Statistical Analysis

All statistical analysis and graphing were processed with OriginPro 2017. All comparisons between two experimental groups were determined using two tailed Welch's t-test, while those between more than two groups were done using one-way ANOVA analysis with Fisher LSD post-hoc test. P-values less than 0.05 were considered significant.

### Example 6: Plural Protease-Triggered In Vitro Drug Released from Modular Conjugate-Based Hydrogel

Typically, we used the following procedure to prepare 20 μL of dual protease-triggered modular conjugate-based hydrogel. Firstly, 10 mg of 8-arm PEG-maleimide (8-PEG-MAL, 40 kDa) was dissolved in 98.33 μL of a PBS buffer solution in a 0.5 ml Eppendorf tube. 0.17 mg MMP-9 sensitive peptide (GPRSLSG-RRCG; SEQ ID NO: 20) linked ibuprofen (Mibu, 1332.7 mmol/mg) was dissolved in 1.67 μL of dimethyl sulfoxide (DMSO) and mixed with the 8-PEG-MAL solution in a stoichiometric ratio of 1:2. After that, 1.62 mg of MMP-9 degradable peptide crosslinker (GCRR-GPRSLSG-RRCG; SEQ ID NO: 21) and 1.87 mg HNE degradable peptide crosslinker (GRCR-PMAVVQSVP-RCRG; SEQ ID NO: 31) were dissolved in 17 μL of the PBS buffer solution separately and mixed in a stoichiometric ratio of 1:1. Final PEG-peptide hydrogels were prepared by reacting the mixture of 8-PEG-MAL and Mibu with the mixture of two peptide crosslinkers in a stoichiometric ratio of 4:1. The final hydrogel solutions were homogenized by vortexing and centrifugation for 3 seconds respectively. Crosslinking time was counted by observation, starting from initial mixing time until there was no free flow solution observed. The hydrogels were also flicked and observed under room light. Formation of the hydrogels were confirmed when transparent and intact hydrogels were observed stick at bottom of the tube and without forming any bubble inside when hydrogel tubes were flicked vigorously. Hydrogels then were used in all subsequent in-vitro gel degradation and drug release experiments.

Briefly, 20 μl of PEG-peptide hydrogels were immersed in 200 μl of PBS solution with or without an enzyme mixture MMP-9 (2 μg/ml) and HNE (1 μg/ml) in a 1.5 mL tube respectively while another two 20 μl of PEG-peptide hydrogels were exposed to the MMP-9-containing HEPES solution (2 μg/ml) or HNE-containing HEPES solution (1 μg/ml) respectively. Each tube was incubated at 37° C. At predetermined time intervals (4, 8, 12, 24, 36, 48 hours), 5 μl aliquot of the liquid sample from each tube was collected and added to 15 μl of buffer solution to dilute it four times in a 2 ml glass vial. A 5 μl volume of the corresponding buffer solution was added to each tube to replace the aliquoted volume. The concentration of digested peptide fragments in all collected samples was quantified by HPLC. The HPLC analysis was conducted at room temperature. The mobile phase used in HPLC was ultra-pure water and acetonitrile at a volume ratio of 35/65 with 0.1% trifluoroacetic acid at a flow rate of 1.0 mL/min. The detected spectrum was converted to drug concentration, which was used to calculate the cumulative percentage of drug release at predetermined time points. The calculation was done by dividing the cumulative amount of drug release (sum of instantaneous drug release and drug loss at previous time points) by the initial amount of peptides used in each hydrogel. The release profile was generated by plotting cumulative percentage drug release versus release time. Data at each time point was obtained from the average of triplicate experiments with a standard deviation.

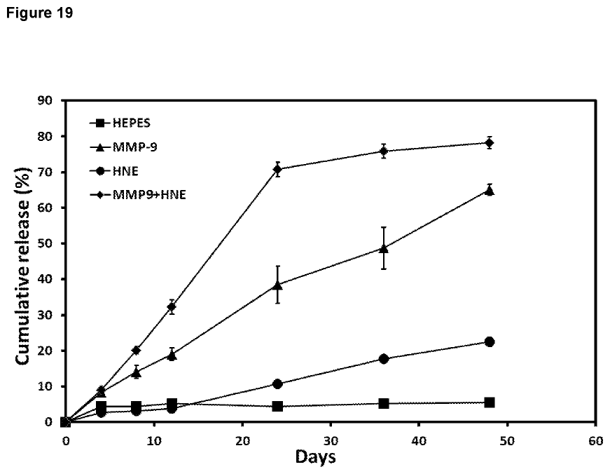

Release kinetics of an anti-inflammatory drug from the hydrogel was investigated in vitro in response to the presence of enzyme solution containing MMP-9 or HNE or MMP-9/HNE mixture. The control experiment was done in pure buffer solution without enzyme. The release profile was obtained by plotting cumulative release at predetermined time points (4, 8, 12, 24, 36, 48 hours after exposure to enzyme solutions), shown in FIG. 19. As shown in FIG. 19, exposing GEP-peptide hydrogel to buffer solution containing MMP-9 and HNE for 48 hours achieved the highest cumulative drug release up to 80%, which was much higher than the cumulative release in fresh buffer solution without any enzyme (7%). When the hydrogel was exposed to a buffer solution containing a single enzyme, MMP-9, the ultimate cumulative release achieved 71%, which was lower but close to maximum cumulative drug release in the mixture of MMP-9 and HNE. When exposing gel to protease-free HEPES buffer, the maximum drug release amount at 48 hours was 9% lower than the ultimate release amount in the MN enzyme mixture solution because intact hydrogel system performed resistance on the diffusion event from the hydrogel and some drug molecules possibly remained stuck inside of the hydrogel matrix due to insufficient diffusion. The same problem was observed when the hydrogel was exposed to enzyme HNE, in which maximum cumulative drug release reached 24%, which was much lower than the release in enzyme MMP-9 solution mainly because enzyme HNE could not cleave drug conjugate like enzyme MMP-9. However, based on drug release profile, small amount of drug molecules could still be cleaved and diffused out from the hydrogel. It was mainly due to hydrogel swelling in an aqueous environment that altered hydrogel structure and cleaved peptide crosslinkers without dissolution.

### SUMMARY

For administration of anti-inflammatory therapeutics, multiple strategies have been attempted to improve spatiotemporal control of drug release kinetics including physically encapsulating drugs or permanently conjugating drugs to polymer backbones [14-17]. However, the release kinetics of these systems could not adapt to changes in the severities of inflammatory diseases. Exploiting enzymes upregulated during arthritic flares as biological cues to activate the drug release, Joshi et al. leveraged the self-assembly of triglycerol monostearate (TG-18) to physically entrap a corticosteroid in a hydrogel platform [20]. However, the drug release from this platform relies on the cleavage of ester bonds on the TG-18 backbone primarily by esterases. Non-enzymatic hydrolysis of these ester bonds in the low pH environment associated with inflammatory conditions [24-26] might also result in undesirable non-specific drug release.

Our invention focused on designing better-performing drug delivery platform with improved control over basal release rate and/or enhanced selectivity and specificity to the inflammation-associated condition. Overall, we have demonstrated several advantageous characteristics of this platform: (1) modular system design consisting of multiple integrating subdomains, each of which possessed a distinct function and could be created and replaced individually to tailor drug loading and drug release kinetics to specific inflammation-associated conditions/diseases by varying the chemical composition of constituent material; (2) the ability to tailor the basal release rate by either significantly minimizing the basal release of drug via covalent conjugation of drugs/modified drugs to the inflammation-responsive hydrogels through protease-cleavable peptides or maintaining some moderate basal release using drug-loaded polymeric particles as the drug-containing domain; (3) the combination of peptide sequences to enable the platform to release loaded cargo upon exposure to one or more disease-specific protease(s), potentially enhancing its specificity to release tailored dosage correlating with the inflammation severity of the disease. These advantages are demonstrated in several examples below.

Modular Particulate-Based Hydrogel with Singular or Plural Protease Responsivity

We developed a modular hybrid hydrogel which could be triggered to release an anti-inflammatory drug upon exposure to elevated protease activity associated with inflammatory diseases. Upon exposure to protease activity, the hydrogel matrix could be proteolytically degraded to liberate the embedded particles and consequently deliver the desired therapeutic payload. Modular design of the hybrid hydrogel enabled independent optimization of its protease-cleavable and drug-loaded subdomains to facilitate hydrogel formation, cleavability by matrix-metalloprotease-9 (MMP-9) to ultimately deliver desirable payload at tunable release rate. In vitro study demonstrated the protease-triggered enhancement of drug release from the hybrid hydrogel system for effective inhibition of TNF-α production by pro-inflammatory macrophages and suggested its potential to mitigate drug-induced cytotoxicity. Using non-invasive imaging to monitor the activity of reactive oxygen species in biomaterial-induced host response, we confirmed that the hybrid hydrogel and its constituent materials did not induce adverse immune response after 5 days following their subcutaneous injection in immuno-competent mice. We subsequently incorporated this hybrid hydrogel onto a commercial wound dressing which could release the drug upon exposure to MMP-9. Together, our findings suggested that this hybrid hydrogel might be a versatile platform for on-demand drug delivery via either injectable or topical application to modulate inflammation in chronic diseases.

Modular Conjugate-Based Hydrogel with Singular or Plural Protease Responsivity

Modular hydrogel systems conjugated with anti-inflammatory drug was also developed. We demonstrated that triggered release of therapeutic drug can be achieved by singular or dual protease stimuli. In some embodiments, the drug loading capacity of the drug-conjugated hydrogel system could be increased by manipulating the configuration of polyethylene glycol which was the hydrogel backbone. The drug release rate was tuned by changing protease-cleavable peptide anchors and crosslinkers. In addition, the in vivo protease-triggered drug release was demonstrated using a model of chemically induced subcutaneous inflammation with different severity levels.

### REFERENCES

Any listing or discussion of an apparently prior-published document in this specification should not necessarily be taken as an acknowledgement that such document is part of the state of the art or is common general knowledge.
Abdelaziza, Hadeer M. et al., Inhalable particulate drug delivery systems for lung cancer therapy: Nanoparticles, microparticles, nanocomposites and nanoaggregates. Journal of Controlled Release 269 (2018) 374-392.
Amano, S. U.; Cohen, J. L.; Vangala, P.; Tencerova, M.; Nicoloro, S. M.; Yawe, J. C.; Shen, Y.; Czech, M. P.; Aouadi, M., Local proliferation of macrophages contributes to obesity-associated adipose tissue inflammation. Cell metabolism 2014, 19 (1), 162-171.
Badeau, B. A.; Comerford, M. P.; Arakawa, C. K.; Shadish, J. A.; DeForest, C. A., Engineered modular biomaterial logic gates for environmentally triggered therapeutic delivery. Nature Chemistry 2018, 10, 251.
Bellocq A., Suberville S., Philippe C., Bertrand F., J. Perez, B. Fouqueray, G. Cherqui, L. Baud, Low Environmental pH Is Responsible for the Induction of Nitric-oxide Synthase in Macrophages: EVIDENCE FOR INVOLVEMENT OF NUCLEAR FACTOR-KB ACTIVATION, Journal of Biological Chemistry 273(9) (1998) 5086-5092 10.1074/jbc.273.9.5086.
Biela, A.; Watkinson, M.; Meier, U. C.; Baker, D.; Giovannoni, G.; Becer, C. R.; Krause, S., Disposable MMP-9 sensor based on the degradation of peptide cross-linked hydrogel films using electrochemical impedance spectroscopy. Biosensors and Bioelectronics 2015, 68, 660-667.
Caley M. P., Martins V. L. C., O'Toole E. A., Metalloproteinases and Wound Healing, Adv Wound Care (New Rochelle) 4(4) (2015) 225-234 http://doi.org/10.1089/wound.2014.0581.
Dang, T. T.; Bratlie, K. M.; Bogatyrev, S. R.; Chen, X. Y.; Langer, R.; Anderson, D. G., Spatiotemporal effects of a controlled-release anti-inflammatory drug on the cellular dynamics of host response. Biomaterials 2011, 32 (19), 4464-4470.
Dang, T. T.; Thai, A. V.; Cohen, J.; Slosberg, J. E.; Siniakowicz, K.; Doloff, J. C.; Ma, M.; Hollister-Lock, J.; Tang, K. M.; Gu, Z.; Cheng, H.; Weir, G. C.; Langer, R.; Anderson, D. G., Enhanced function of immuno-isolated islets in diabetes therapy by co-encapsulation with an anti-inflammatory drug. Biomaterials 2013, 34 (23), 5792-5801.
Friedman, M.; Cavins, J. F.; Wall, J. S., Relative Nucleophilic Reactivities of Amino Groups and Mercaptide Ions in Addition Reactions with α,β-Unsaturated Compounds1, 2. Journal of the American Chemical Society 1965, 87 (16), 3672-3682.
Han, F. Y.; Thurecht, K. J.; Whittaker, A. K.; Smith, M. T., Bioerodable PLGA-Based Microparticles for Producing Sustained-Release Drug Formulations and Strategies for Improving Drug Loading. Frontiers in pharmacology 2016, 7, 185-185.
Hämäläinen M., Nieminen R., Uurto I., Salenius J.-P., Kellomaki M., Mikkonen J., Kotsar A., Isotalo T., Lj T. T., Talja M., Moilanen E., Dexamethasone-eluting Vascular Stents, Basic & Clinical Pharmacology & Toxicology 112(5) (2013) 296-301 http://doi.org/10.1111/bcpt.12056.
Hsu B. B., Park M.-H., Hagerman S. R., Hammond P. T., Multimonth controlled small molecule release from biodegradable thin films, Proceedings of the National Academy of Sciences 111(33) (2014) 12175 https://doi.org/10.1073/pnas.1323829111.
Jones, B. H.; Martinez, A. M.; Wheeler, J. S.; Spoerke, E. D., Surfactant-induced assembly of enzymatically-stable peptide hydrogels. Soft Matter 2015, 11 (18), 3572-3580.
Joshi N., Yan J., Levy S., Bhagchandani S., Slaughter K. V., Sherman N. E., Amirault J., Wang Y., Riegel L., He X., Rui T. S., Valic M., Vemula P. K., Miranda 0. R., Levy O., Gravallese E. M., Aliprantis A. O., Ermann J., Karp J. M., Towards an arthritis flare-responsive drug delivery system, Nature Communications 9(1) (2018) 1275 http://doi.org/10.1038/s41467-018-03691-1.
Ladwig G. P., Robson M. C., Liu R. A. N., Kuhn M. A., Muir D. F., Schultz G. S., Ratios of activated matrix metalloproteinase-9 to tissue inhibitor of matrix metalloproteinase-1 in wound fluids are inversely correlated with healing of pressure ulcers, Wound Repair and Regeneration 10(1) (2002) 26-37 http://doi.org/10.1046/j. 1524-475X.2002. 10903.x.
Li, J.; Mooney, D. J., Designing hydrogels for controlled drug delivery. Nature Reviews Materials 2016, 1, 16071.
Liu, W. F.; Ma, M.; Bratlie, K. M.; Dang, T. T.; Langer, R.; Anderson, D. G., Real-time in vivo detection of biomaterial-induced reactive oxygen species. Biomaterials 2011, 32 (7), 1796-1801.
Lutolf, M. P.; Tirelli, N.; Cerritelli, S.; Cavalli, L.; Hubbell, J. A., Systematic Modulation of Michael-Type Reactivity of Thiols through the Use of Charged Amino Acids. Bioconjugate Chemistry 2001, 12 (6), 1051-1056.
Lutolf, M. P.; Hubbell, J. A., Synthesis and Physicochemical Characterization of End-Linked Poly(ethylene glycol)-co-peptide Hydrogels Formed by Michael-Type Addition. Biomacromolecules 2003, 4 (3), 713-722.
Pham C. T. N., Neutrophil serine proteases fine-tune the inflammatory response, The International Journal of Biochemistry & Cell Biology 40(6) (2008) 1317-1333 https://doi.org/10.1016/j.biocel.2007.11.008.
Roomi, M. W.; Monterrey, J. C.; Kalinovsky, T.; Rath, M.; Niedzwiecki, A., Distinct patterns of matrix metalloproteinase-2 and -9 expression in normal human cell lines. Oncology reports 2009, 21 (3), 821-6.
Roos, G.; Foloppe, N.; Messens, J., Understanding the pKa of Redox Cysteines: The Key Role of Hydrogen Bonding. Antioxidants & Redox Signaling 2012, 18 (1), 94-127.
Samuelson, L. E.; Scherer, R. L.; Matrisian, L. M.; McIntyre, J. O.; Bornhop, D. J., Synthesis and In Vitro Efficacy of MMP9-Activated NanoDendrons. Molecular Pharmaceutics 2013, 10 (8), 3164-3174.
Seong, S.-Y.; Matzinger, P., Hydrophobicity: an ancient damage-associated molecular pattern that initiates innate immune responses. Nature Reviews Immunology 2004, 4, 469.
Serhan C. A., Ward P. A., Gilroy D. W., Fundamentals of Inflammation, Cambridge University Press, Cambridge, 2010.
Steinmeyer, J., Pharmacological basis for the therapy of pain and inflammation with nonsteroidal anti-inflammatory drugs. Arthritis Research & Therapy 2000, 2 (5), 379.
Sutherland, E. R.; Allmers, H.; Ayas, N. T.; Venn, A. J.; Martin, R. J., Inhaled corticosteroids reduce the progression of airflow limitation in chronic obstructive pulmonary disease: a meta-analysis. Thorax 2003, 58 (11), 937-941.
Tomei, A. A.; Manzoli, V.; Fraker, C. A.; Giraldo, J.; Velluto, D.; Najjar, M.; Pileggi, A.; Molano, R. D.; Ricordi, C.; Stabler, C. L.; Hubbell, J. A., Device design and materials optimization of conformal coating for islets of Langerhans. Proceedings of the National Academy of Sciences 2014, 111 (29), 10514.
Youssef J., Novosad S. A., Winthrop K. L., Infection Risk and Safety of Corticosteroid Use, Rheumatic diseases clinics of North America 42(1) (2016) 157-176 http://doi.org/10.1016/j.rdc.2015.08.004.
Zhou, Q.; Lin, J.; Wang, J.; Li, F.; Tang, F.; Zhao, X., A designed amphiphilic peptide containing the silk fibroin motif as a potential carrier of hydrophobic drugs. Progress in Natural Science 2009, 19 (11), 1529-1536.
Zoia, C.; Bongetta, D.; Lombardi, F.; Custodi, V. M.; Pugliese, R.; Gaetani, P., First Impressions about Adherus, a New Dural Sealant. Journal of Applied Biomaterials & Functional Materials 2015, 13 (4), 372-375.

1. A drug-loaded protease-responsive hydrogel comprising;
a) a drug encapsulated in particles;
b) a polymer building block comprising of a multi-arm-polyethylene glycol (PEG) with functional moiety; and
c) a bis-functional protease-sensitive crosslinker comprising a protease-cleavable substrate flanked by two spacer sequences containing functional moieties;
wherein said polymer building block of b) forms a gel in the presence of the protease-cleavable crosslinker of c) to entrap the particles of a).

2. The drug-loaded protease-responsive hydrogel of claim 1, further comprising:
a) at least a second bis-functional protease-sensitive crosslinker which comprises a protease-cleavable substrate, sensitive to a different protease to that of said crosslinker of c), flanked by spacer sequences containing functional moieties; and/or
b) at least a further bis-functional protease-resistant crosslinker which comprises a protease-resistant substrate.

3. The drug-loaded protease-responsive hydrogel of claim 1 or 2, wherein the drug is encapsulated in particles comprising a material selected from the group comprising silica, liposomes, siRNA complexes and polymeric materials such as polycaprolactone, poly (methacrylic acids), polylactic acids, polyvinylpirrolidone, poly(lactic-co-glycolic acid) (PLGA), and gelatin.

4. The drug-loaded protease-responsive hydrogel of any one of claims 1 to 3, wherein the polymer building block comprises a multi-arm-PEG-vinyl sulfone or multi-arm-PEG-maleimide or multi-arm-PEG-azide or multi-arm-PEG-alkyne.

5. A drug-loaded protease-responsive hydrogel comprising;
a) a drug covalently conjugated to a protease-cleavable peptide anchor having a functional moiety;
b) a polymer building block comprising a multi-arm-PEG polymer having at least one functional moiety; and
c) a bis-functional crosslinker comprising a peptide substrate flanked by spacer sequences containing functional moieties;
wherein the functional moiety of the peptide anchor covalently links the drug to an arm of the multi-arm PEG polymer, and wherein a functional moiety of said polymer building block covalently links to said moiety of said bis-functional crosslinker to form a gel.

6. The drug-loaded protease-responsive hydrogel of claim 5, wherein:
a) said crosslinker is not cleavable to a protease; or
b) said peptide anchor is cleavable to a protease and said crosslinker is cleavable to the same or different protease and/or
c) said drug-loaded hydrogel comprises a plurality of crosslinkers, one or more of which are cleavable to different proteases.

7. The drug-loaded protease-responsive hydrogel of claim 5 or 6, wherein the polymer building block comprises a multi-arm-PEG-vinyl sulfone or a multi-arm-PEG-vinyl Maleimide or a multi-arm-PEG-azide or a multi-arm-PEG-alkyne.

8. The drug-loaded protease-responsive hydrogel of claim 7, comprising 2-12 wt % multi-arm-PEG-vinyl Maleimide.

9. The drug-loaded protease-responsive hydrogel of any one of claims 1 to 8, wherein the multi-arm PEG polymer has 3 to 8 arms.

10. The drug-loaded protease-responsive hydrogel of any one of claims 1 to 9, wherein the drug is anti-inflammatory.

11. The drug-loaded protease-responsive hydrogel of claim 10, wherein the drug is a non-steroidal anti-inflammatory drug (NSAID).

12. The drug-loaded protease-responsive hydrogel of claim 10 or 11, wherein the protease is upregulated during inflammation and is selected from the group comprising matrix metalloproteinases, serine proteases, cysteine proteases and aspartic proteases.

13. The drug-loaded protease-responsive hydrogel of any one of claims 1 to 12, wherein the said flanking spacer sequences comprise at least one Cysteine and/or Lysine residue and/or azide- or alkyne-containing unnatural amino acid.

14. The drug-loaded protease-responsive hydrogel of claim 13, wherein the said flanking spacer sequences comprises a sequence of 1-6 amino acids.

15. The drug-loaded protease-responsive hydrogel of any one of claims 1 to 14, wherein the protease-cleavable substrate is sensitive to a protease selected from the group comprising matrix metalloproteinase, such as metalloproteinase-9 (MMP-9), MMP-2, MMP-7, MMP-12 etc., cathepsins, such as Cathepsin K, Cathepsin B, Cathepsin S, etc., and human neutrophil elastase (HNE), caspases and urokinases.

16. The drug-loaded protease-responsive hydrogel of claim 15, wherein the protease-cleavable substrate is selected from the group comprising MMP-9 substrates comprising the amino acid sequence set forth in KGPRSLSGK (SEQ ID NO: 30), GPRSLSG (SEQ ID NO: 10), LGRMGLPGK (SEQ ID NO: 11), AVRWLLTA (SEQ ID NO: 12) or GPQGIWGQ (SEQ ID NO: 13; HNE substrates comprising APEEIMDRQ (SEQ ID NO: 14) or PMAVVQSVP (SEQ ID NO: 15; Cathepsin B substrates comprising GRRGLG (SEQ ID NO: 16) or DGFLGDD (SEQ ID NO: 17) or a combination thereof.

17. A composition comprising the drug-loaded protease-responsive hydrogel of any one of claims 1 to 16 formulated for injection or topical administration.

18. A dressing comprising the drug-loaded protease-responsive hydrogel of any one of claims 1 to 16.

19. Use of the drug-loaded protease-responsive hydrogel of any one of claims 1 to 16 or a composition of claim 17 as an injectable or topical dressing for treating a subject in need thereof.

20. A method of treatment comprising administering to a subject in need of such treatment an efficacious amount of the drug-loaded protease-responsive hydrogel of any one of claims 1 to 16 or a composition of claim 17.

21. A kit comprising:
a) a drug encapsulated in particles;
b) a polymer building block comprising of a multi-arm-polyethylene glycol (PEG) with functional moiety; and
c) a bis-functional protease-sensitive crosslinker comprising a protease-cleavable substrate flanked by two spacer sequences containing functional moieties,
wherein a)-c) are as defined in any one of the previous claims; or
a) a drug covalently conjugated to a protease-cleavable peptide anchor having a functional moiety;
b) a polymer building block comprising a multi-arm-PEG polymer having at least one functional moiety; and
c) a bis-functional crosslinker comprising a peptide substrate flanked by spacer sequences containing functional moieties,
wherein a)-c) are as defined in any one of the previous claims.

22. A method of manufacturing a drug-loaded protease-responsive hydrogel comprising the steps:
a) mixing a polymer building block comprising of a multi-arm-polyethylene glycol (PEG) with functional moiety with drug-loaded particles;
b) mixing a bis-functional protease-sensitive crosslinker comprising a protease-cleavable substrate flanked by spacer sequences containing functional moieties with drug-loaded particles;
c) mixing together the mixtures of a) and b);
wherein said polymer building block of a) forms a gel in the presence of the protease-cleavable crosslinker of b) to entrap the drug-loaded particles.

23. A method of manufacturing a drug-loaded protease-responsive hydrogel comprising the steps:
a) mixing a drug covalently conjugated to a peptide anchor having a functional moiety with a polymer building block comprising a multi-arm-PEG polymer having at least one functional moiety, wherein the respective functional moieties of the peptide anchor and multi-arm-PEG polymer covalently bond to conjugate the drug to an arm of the multi-arm PEG polymer;
b) mixing the drug-polymer conjugate of a) with a bis-functional crosslinker comprising a peptide substrate flanked by spacer sequences containing functional moieties;
wherein a functional moiety of said polymer building block covalently links to said moiety of said bis-functional crosslinker to form a gel.

24. The method of claim 23, wherein:
a) said peptide anchor is cleavable to a protease and said crosslinker is not cleavable to a protease; or
b) said peptide anchor is cleavable to a protease and said crosslinker is cleavable to the same or different protease; and/or
c) said drug-loaded hydrogel comprises a plurality of crosslinkers, one or more of which are cleavable to different proteases.

25. The method of any one of claims 22 to 24, wherein the drug, the polymeric particles, the crosslinker, the cleavable anchor and/or the polymer building block are as defined in any one of claims 1 to 16.

26. A method of manufacturing a composite dressing comprising a drug-loaded protease-responsive hydrogel of any one of claims 1 to 16, comprising the steps;
a) preparing a mixture of a drug-encapsulated in particles and a bis-functional protease-sensitive crosslinker comprising a protease-cleavable substrate flanked by two spacer sequences containing functional moieties;
b) preparing a mixture of a drug-encapsulated in particles and a polymer building block comprising of a multi-arm-polyethylene glycol (PEG) with functional moiety;
c) mixing a) and b) together, depositing the mixture onto a dressing and allowing gelation.

27. The method of claim 26, wherein the dressing is an alginate wound dressing.

28. The method of claim 26 or 27 further comprising step d), wherein the composite dressing is flash-frozen in liquid nitrogen and lyophilized to dryness.

29. The method of any one of claims 22 to 28, wherein the drug is a NSAID; the particle comprises poly(lactic-co-glycolic acid) (PLGA); the crosslinker and/or anchor are cleavable to a protease selected from the group comprising matrix metalloproteinases and serine proteases or combinations thereof; and the polymer building block comprises a 4- or 8-arm-PEG-vinyl sulfone or a 4- or 8-arm-PEG-vinyl Maleimide or a 4- or 8-arm-PEG-azide or a 4- or 8-arm-PEG-alkyne.


SEQUENCE LISTING (40 sequences)

SEQ ID NO: 1 - protein, 4 aa, Artificial Sequence
  GRCR Spacer
GRCR

SEQ ID NO: 2 - protein, 4 aa, Artificial Sequence
  GCRG spacer
GCRG

SEQ ID NO: 3 - protein, 4 aa, Artificial Sequence
  GRCD spacer
GRCD

SEQ ID NO: 4 - protein, 4 aa, Artificial Sequence
  GCDR spacer
GCDR

SEQ ID NO: 5 - protein, 4 aa, Artificial Sequence
  GCDG spacer
GCDG

SEQ ID NO: 6 - protein, 4 aa, Artificial Sequence
  GDCD spacer
GDCD

SEQ ID NO: 7 - protein, 4 aa, Artificial Sequence
  GCDD spacer
GCDD

SEQ ID NO: 8 - protein, 4 aa, Artificial Sequence
  GCRD spacer
GCRD

SEQ ID NO: 9 - protein, 4 aa, Artificial Sequence
  GCRR spacer
GCRR

SEQ ID NO: 10 - protein, 7 aa, Artificial Sequence
  GPRSLSG substrate
GPRSLSG

SEQ ID NO: 11 - protein, 9 aa, Artificial Sequence
  LGRMGLPGK substrate
LGRMGLPGK

SEQ ID NO: 12 - protein, 8 aa, Artificial Sequence
  AVRWLLTA substrate
AVRWLLTA

SEQ ID NO: 13 - protein, 8 aa, Artificial Sequence
  GPQGIWGQ substrate
GPQGIWGQ

SEQ ID NO: 14 - protein, 9 aa, Artificial Sequence
  APEEIMDRQ substrate
APEEIMDRQ

SEQ ID NO: 15 - protein, 9 aa, Artificial Sequence
  PMAVVQSVP substrate
PMAVVQSVP

SEQ ID NO: 16 - protein, 6 aa, Artificial Sequence
  GRRGLG substrate
GRRGLG

SEQ ID NO: 17 - protein, 7 aa, Artificial Sequence
  DGFLGDD substrate
DGFLGDD

SEQ ID NO: 18 - protein, 17 aa, Artificial Sequence
  GCRR-KGPRSLSGK-RRCG
GCRRKGPRSLSGKRRCG

SEQ ID NO: 19 - protein, 12 aa, Artificial Sequence
  GPQGIWGQ-DRCG
GPQGIWGQDRCG

SEQ ID NO: 20 - protein, 11 aa, Artificial Sequence
  GPRSLSGRRCG
GPRSLSGRRCG

SEQ ID NO: 21 - protein, 15 aa, Artificial Sequence
  GCRR-GPRSLSG-RRCG
GCRRGPRSLSGRRCG

SEQ ID NO: 22 - protein, 16 aa, Artificial Sequence
  GCRD-GPQGIWGQ-DRCG
GCRDGPQGIWGQDRCG

SEQ ID NO: 23 - protein, 17 aa, Artificial Sequence
  GCRR-LGRMGLPGK-RRCG
GCRRLGRMGLPGKRRCG

SEQ ID NO: 24 - protein, 16 aa, Artificial Sequence
  GCRR-AVRWLLTA-RRCG
GCRRAVRWLLTARRCG

SEQ ID NO: 25 - protein, 16 aa, Artificial Sequence
  GCRR-GPQGIWGQ-RRCG
GCRRGPQGIWGQRRCG

SEQ ID NO: 26 - protein, 17 aa, Artificial Sequence
  GCRD-LGRMGLPGK-DRCG
GCRDLGRMGLPGKDRCG

SEQ ID NO: 27 - protein, 17 aa, Artificial Sequence
  GCRD-KGPRSLSGK-DRCG
GCRDKGPRSLSGKDRCG

SEQ ID NO: 28 - protein, 16 aa, Artificial Sequence
  GCRD-AVRWLLTA-DRCG
GCRDAVRWLLTADRCG

SEQ ID NO: 29 - protein, 17 aa, Artificial Sequence
  GCRR-KSSRGGPLK-RRCG
GCRRKSSRGGPLKRRCG

SEQ ID NO: 30 - protein, 9 aa, Artificial Sequence
  KGPRSLSGK
KGPRSLSGK

SEQ ID NO: 31 - protein, 17 aa, Artificial Sequence
  GRCR-PMAVVQSVP-RCRG
GRCRPMAVVQSVPRCRG

SEQ ID NO: 32 - protein, 15 aa, Artificial Sequence
  GRCR-GPRSLSG-RCRG
GRCRGPRSLSGRCRG

SEQ ID NO: 33 - protein, 4 aa, Artificial Sequence
  GXXX Spacer seqiuence
  MISC_FEATURE (2)..(4): Xaa is independently Glycine, Cysteine, Aspartic acid, or Arginine.
GXXX

SEQ ID NO: 34 - protein, 4 aa, Artificial Sequence
  XXXG
  MISC_FEATURE (1)..(3): Xaa is independently Glycine, Cysteine, Aspartic acid, or Arginine.
XXXG

SEQ ID NO: 35 - protein, 17 aa, Artificial Sequence
  GRCR-APEEIMDRQ-RCRG
GRCRAPEEIMDRQRCRG

SEQ ID NO: 36 - protein, 16 aa, Artificial Sequence
  GRCR-GPQGIWGQ-RCRG
GRCRGPQGIWGQRCRG

SEQ ID NO: 37 - protein, 14 aa, Artificial Sequence
  GRCR-GRRGLG-RCRG
GRCRGRRGLGRCRG

SEQ ID NO: 38 - protein, 15 aa, Artificial Sequence
  GRCR- DGFLGDD-RCRG
GRCRDGFLGDDRCRG

SEQ ID NO: 39 - protein, 15 aa, Artificial Sequence
  xM scrambled
GCRRSSRGGPLRRCG

SEQ ID NO: 40 - protein, 11 aa, Artificial Sequence
  M scrambled
SSRGGPLRRCG